(19) United States
(12) Patent Application Publication
(10) Pub. No.: US 2024/0376525 A1
(43) Pub. Date: Nov. 14, 2024

(54) USE OF ETHYLENE CARBONATE IN NUCLEIC ACID SEQUENCING METHODS

(71) Applicant: Ultima Genomics, Inc., Fremont, CA (US)

(72) Inventors: Xi LONG, Fremont, CA (US); Florian OBERSTRASS, Redwood City, CA (US)

(21) Appl. No.: 18/763,834

(22) Filed: Jul. 3, 2024

Related U.S. Application Data
(63) Continuation of application No. PCT/US2023/060778, filed on Jan. 17, 2023.
(60) Provisional application No. 63/300,343, filed on Jan. 18, 2022.

(51) Int. Cl. C12Q 1/6806; C12Q 1/6844; C12Q 1/6869; C12Q 1/6876

(52) U.S. Cl. CPC C12Q 1/6806; C12Q 1/6844; C12Q 1/6869; C12Q 1/6876

(57) ABSTRACT

Provided herein are methods and are methods for preparing nucleic acid molecules for sequencing on a surface using ethylene carbonate. Further described are methods for sequencing said nucleic acid molecules on a surface, using flow sequencing methods.

### CROSS-REFERENCE TO RELATED APPLICATIONS

This application is a continuation application of International Application No. PCT/US2023/060778, filed Internationally on Jan. 17, 2023, which claims priority to and the benefit of U.S. Provisional Patent App. No. 63/300,343, filed Jan. 18, 2022, which are incorporated herein by reference herein in their entirety.

### FIELD

Described herein are methods for preparing nucleic acid molecules for sequencing using ethylene carbonate. Further described are methods for sequencing said nucleic acid molecules.

### BACKGROUND

Next-generation sequencing (NGS) has provided researchers and clinical laboratories the tools needed to simultaneously sequence many different nucleic acid molecules in a single sample. Certain highly efficient NGS methods utilize non-terminating nucleotides to sequence nucleic acid molecules. These sequencing methods may be referred to as “flow sequencing,” “natural sequencing-by-synthesis,” or “non-terminated sequencing-by-synthesis” methods (see, for example, U.S. Pat. No. 8,772,473, which is incorporated herein by reference in its entirety). In order to prepare for sequencing, nucleic acid molecules undergo a series of steps, often including on-surface amplification followed by hybridization of sequencing primers, processes which are often not thermally compatible. Furthermore, nucleic acid molecules may require denaturing to generate single-stranded molecules prior to hybridization of the sequencing primers, thus requiring longer workflow periods.

### BRIEF SUMMARY

Methods for preparing nucleic acid molecules for sequencing are described herein. The methods can include the use of ethylene carbonate for denaturing double-stranded nucleic acid molecules attached to a surface, which can allow a sequencing primer to hybridize to the resulting single-stranded nucleic acid molecule. In addition, the use of ethylene carbonate can combine the denaturation of duplex nucleic acid and the hybridization of sequencing primer into a single step. For example, the concentration of sequencing primer may be selected to favor the formation of sequencing hybrids (e.g., excess concentration of sequencing primer), and/or the sequencing primer may be designed to increase the stability of the sequencing hybrids (e.g., the sequencing primer comprises peptide nucleic acids (PNAs)). Also provided are methods for sequencing the nucleic acid molecules following the use of the nucleic acid molecule sequencing preparation methods described herein. The methods can be used, for example, to shorten the workflow of on-surface amplification of nucleic acid molecules. Furthermore, the methods of preparing nucleic acid molecules may be isothermal with nucleic acid molecule amplification (e.g., recombinase polymerase amplification) and downstream sequencing (e.g., polymerase chain reaction).

In some aspects, provided herein is a method of preparing nucleic acid molecules for sequencing, comprising: contacting double-stranded nucleic acid molecules attached to a surface with ethylene carbonate to generate single-stranded nucleic acid molecules attached to the surface; and, hybridizing sequencing primers to the single-stranded nucleic acid molecules, thereby generating sequencing hybrids. In some embodiments, the ethylene carbonate has a concentration of between about 10% and about 50% volume/volume. In some embodiments, the contacting is implemented at a temperature of between about 35° C. and about 50° C. In some embodiments, the double-stranded nucleic acid molecules attached to the surface are contacted with the ethylene carbonate for about 5 minutes or more.

In some embodiments, the sequencing primer is a nucleic acid primer. In some embodiments, the sequencing primer is a peptide nucleic acid (PNA) primer. In some embodiments, the PNA primer has increased hybridization affinity with the single-strand nucleic acid molecules compared to a nucleic acid primer. In some embodiments, the sequencing primer concentration is selected so that the hybridization reaction equilibrium favors the formation of sequencing hybrids. In some embodiments, the sequencing primers are in excess concentration compared to the single-stranded nucleic acid molecules. In some embodiments, the contacting and hybridizing occur simultaneously.

In some embodiments, the double-stranded nucleic acid molecules or the single-stranded nucleic acid molecules are derived from a fluidic sample obtained from an individual. In some embodiments, the fluidic sample is a blood sample, a plasma sample, a saliva sample, a urine sample, or a fecal sample. In some embodiments, the fluidic sample comprises cell-free nucleic acid molecules. In some embodiments, the fluidic sample comprises DNA molecules. In some embodiments, the fluidic sample comprises cDNA molecules.

In some embodiments, the double-stranded nucleic acid molecules or the single-stranded nucleic acid molecules comprise a sequencing adaptor sequence, wherein the sequencing adaptor sequence comprises a sequencing primer hybridization sequence. In some embodiments, the double-stranded nucleic acid molecules or the single-stranded nucleic acid molecules are amplification products.

In some embodiments, the double-stranded nucleic acid molecules or the single-stranded nucleic acid molecules are covalently attached to the surface. In some embodiments, the double-stranded nucleic acid molecules or the single-stranded nucleic acid molecules are attached to the surface using click chemistry or amine-reactive crosslinker chemistry. In some embodiments, the surface is a bead. In some embodiments, the bead is a gel bead. In some embodiments, the surface is immobilized to a wafer.

In some embodiments, the method further comprises attaching nucleic acid molecules in a sequencing library to the surface prior to the contacting with ethylene carbonate. In some embodiments, the method further comprises amplifying the nucleic acid molecules in the sequencing library attached to the surface prior to the contacting with ethylene carbonate, thereby generating sequencing colonies comprising the double-stranded nucleic acid molecules attached to the surface. In some embodiments, the nucleic acid molecules are amplified isothermally. In some embodiments, the amplifying occurs between about 30° C. and about 50° C. In some embodiments, the amplifying comprises one or more of rolling circle amplification (RCA), multiple displacement amplification (MDA), recombinase polymerase amplification (RPA), and molten recombinase polymerase amplification (mRPA). In some embodiments, the amplifying comprises use of reagents selected from the group consisting of polymerases, recombinases, single-stranded DNA binding proteins, magnesium acetate, betaine, formamide, tetramethyl ammonium chloride, sodium dodecyl sulfate (SDS), and trimethylamine N-oxide. In some embodiments, the method further comprises removing deoxyuridine primers after amplifying the nucleic acid molecules on the surface.

In some embodiments, the method further comprises washing the single-stranded nucleic acid molecules with a wash buffer prior to the hybridizing of sequencing primers to the single-stranded nucleic acid molecules. In some embodiments, the method further comprises washing the sequencing hybrids with a wash buffer. In some embodiments, the washing is repeated two or more times. In some embodiments, the wash buffer comprises tris(hydroxymethyl)aminomethane (tris), ethylenediaminetetraacetic acid (EDTA), triton, or sodium dodecyl sulfate (SDS).

In some embodiments, the method further comprises sequencing the single-stranded nucleic acid molecules, thereby generating sequencing data. In some embodiments, the single-stranded nucleic acid molecules are sequenced using a plurality of sequencing flow steps, each sequencing flow step comprising contacting the sequencing hybrids with nucleotides, wherein at least a portion of the nucleotides are labeled, and detecting the presence or absence of an incorporated nucleotide. In some embodiments, the nucleotides in each sequencing flow step comprise nucleotides of a same base type. In some embodiments, the at least a portion of the nucleotides is less than all of the nucleotides in each sequencing flow step. In some embodiments, the nucleotides are non-terminating nucleotides. In some embodiments, the sequencing data comprises flow signals at the plurality of sequencing flow steps. In some embodiments, the flow signals are used to determine a base count indicative of a number of bases sequenced at each flow step. In some embodiments, the flow signals are used to determine a statistical parameter indicative of a likelihood for at least one base count at each flow step, wherein the base count is indicative of a number of bases of a given single-stranded nucleic acid molecule sequenced at a given flow step.

In some aspects, provided herein is a method of preparing nucleic acid molecules for sequencing, comprising: providing nucleic acid molecules attached to a surface; amplifying the nucleic acid molecules on the surface to generate double-stranded nucleic acid molecules; contacting the double-stranded nucleic acid molecules with ethylene carbonate to generate single-stranded nucleic acid molecules; washing the single-stranded nucleic acid molecules with a wash buffer; and, hybridizing sequencing primers to the single-stranded nucleic acid molecules, thereby generating sequencing hybrids. In some embodiments, the amplifying is isothermal. In some embodiments, the amplifying comprises one or more of rolling circle amplification (RCA), multiple displacement amplification (MDA), recombinase polymerase amplification (RPA), and molten recombinase polymerase amplification (mRPA).

In some aspects, provided herein is a method of sequencing nucleic acid molecules, comprising: providing nucleic acid molecules attached to a surface; amplifying the nucleic acid molecules on the surface to generate double-stranded nucleic acid molecules; contacting the double-stranded nucleic acid molecules with ethylene carbonate to generate single-stranded nucleic acid molecules; washing the single-stranded nucleic acid molecules with a wash buffer; hybridizing sequencing primers to the single-stranded nucleic acid molecules, thereby generating sequencing hybrids; and, sequencing the single-stranded nucleic acid molecules using a plurality of sequencing flow steps, each sequencing flow step comprising contacting the sequencing hybrids with non-terminating nucleotides, wherein at least a portion of the non-terminating nucleotides are labeled, and detecting the presence or absence of an incorporated non-terminating nucleotide. In some embodiments, the amplifying is isothermal. In some embodiments, the amplifying comprises one or more of rolling circle amplification (RCA), multiple displacement amplification (MDA), recombinase polymerase amplification (RPA), and molten recombinase polymerase amplification (mRPA). In some embodiments, the method further comprises generating sequencing data, wherein the sequencing data comprises flow signals detected at the plurality of sequencing flow steps. In some embodiments, the flow signals are used to determine a base count indicative of a number of bases sequenced at each flow step. In some embodiments, the flow signals are used to determine a statistical parameter indicative of a likelihood for at least one base count at each flow step, wherein the base count is indicative of a number of bases of a given single-stranded nucleic acid molecule sequenced at a given flow step.

### BRIEF DESCRIPTION OF THE DRAWINGS

FIG. 1 shows an exemplary workflow for preparing nucleic acid molecules for sequencing on a surface, in accordance with some embodiments.

FIG. 2 shows an exemplary workflow for next-generation sequencing of nucleic acid molecules, in accordance with some embodiments.

FIG. 3 shows an exemplary quantification of denaturation of nucleic acid molecules on a surface, following treatment with sodium hydroxide (NaOH) or ethylene carbonate (EC).

FIG. 4A shows example data on denaturation of nucleic acid molecules on a surface following control treatment (no NaOH or EC). FIG. 4B shows example data on denaturation of nucleic acid molecules on a surface following treatment with 100 mM NaOH. FIG. 4C shows example data on denaturation of nucleic acid molecules on a surface following treatment with ethylene carbonate (EC). Nucleic acid molecules in FIGS. 4A-4C are about 200˜300 bp in length.

FIG. 5A shows example data on sequencing primer hybridization, in presence of ethylene carbonate (EC). FIG. 5B shows the coupon shown in FIG. 5A after subsequent NaOH treatment.

FIG. 6 shows example data on the location of sequencing primers hybridized to amplicons in the presence of ethylene carbonate (EC).

### DETAILED DESCRIPTION

Next-generation sequencing methods of nucleic acid molecules have provided the ability to generate significant amounts of data regarding various nucleic acid molecules in a single sample. The preparation of nucleic acid molecules for sequencing at such a high depth can be time consuming, and more efficient library preparation methods can significantly improve sequencing throughput. Provided herein are methods for preparing nucleic acid molecules (e.g., double-stranded nucleic acid molecules) attached to a surface for synthesis using ethylene carbonate to generate single-stranded nucleic acid molecules capable of hybridizing with sequencing primers, resulting in a streamlined workflow for analysis (e.g., downstream sequencing) of the nucleic acid molecules.

The nucleic acid molecules according to the present disclosure can be prepared for sequencing isothermally, e.g., at a constant temperature, using ethylene carbonate. In some embodiments, the double-stranded nucleic acid molecules attached to a surface (e.g., a solid support, such as a bead, which may be attached to a wafer sequencing surface) are contacted with ethylene carbonate to generate single-stranded nucleic acid molecules. Ethylene carbonate may convert double-stranded nucleic acid molecules and partially double-stranded nucleic acid molecules into single-stranded nucleic acid molecules. In contrast with other frequently used solvents, ethylene carbonate is non-toxic (e.g., compatible with the surface on which the double-stranded nucleic acids are attached) and does not require heat to denature nucleic acid molecules. Thus, double-stranded nucleic acid molecules attached to a surface, such as any of the double-stranded nucleic acid molecules and surfaces provided herein, may be contacted with ethylene carbonate isothermally with upstream amplification and/or downstream sequencing analysis, to result in a shorter, more streamlined, workflow. For example, in some embodiments the double-stranded nucleic acids attached to the surface are contacted with ethylene carbonate at a temperature of between about 35° C. and about 50° C. In some embodiments, the double-stranded nucleic acids attached to the surface are contacted with ethylene carbonate at room temperature. In some embodiments, the ethylene carbonate is provided at a concentration of between about 20% and about 50% volume/volume. In some embodiments, the ethylene carbonate is provided at a concentration of about 35% volume/volume. In some embodiments, the double-stranded nucleic acid molecules attached to the surface are contacted with the ethylene carbonate for carbonate for about 5 minutes or more. In some embodiments, the double-stranded nucleic acid molecules attached to the surface are contacted with the ethylene carbonate for carbonate for up to about 1 hour. In some embodiments, the double-stranded nucleic acid molecules attached to the surface are contacted with the ethylene carbonate for about 30 minutes.

The nucleic acid molecules (e.g., the double-stranded nucleic acid molecules and/or the single-stranded nucleic acid molecules) may comprise a sequencing adaptor sequence, wherein the sequencing adaptor sequence comprises a sequencing primer hybridization sequence. In some embodiments, the sequencing primers hybridize with the sequencing adaptor sequence, or a portion thereof, of the single-stranded nucleic acid molecules, thereby generating sequencing hybrids. The sequencing primers may be used to sequence (e.g., by a flow sequencing method) the single-stranded nucleic acid molecules, thereby generating sequencing data.

The methods provided herein may further comprise attaching nucleic acid molecules in a sequencing library to a surface prior to contacting with ethylene carbonate. In some embodiments, the nucleic acid molecules in a sequencing library are double-stranded nucleic acids that may be attached to the surface. In some embodiments, the nucleic acid molecules in a sequencing library are amplification products. In some embodiments, a portion of the nucleic acid molecules in a sequencing library are amplification products.

In some embodiments, the double-stranded nucleic acid molecules and/or the single-stranded nucleic acid molecules attached to a surface may be amplification products. Therefore, in some aspects, the methods provided herein further comprise amplifying the nucleic acid molecules attached to the surface, e.g., generating sequencing colonies. The amplification may not require thermal melting (e.g., thermocycling) or chemical melting (e.g., applications of chemical solvents, such as dimethyl sulfoxide (DMSO) to disrupt secondary structure) of the template nucleic acid, and/or may not require the use of thermophilic enzymes. In some embodiments, the nucleic acid molecules are amplified isothermally. In some embodiments, the isothermal amplification occurs between about 30° C. and about 50° C. In some embodiments, the amplifying comprises recombinase polymerase amplification (RPA) or molten recombinase polymerase amplification (RPA).

In some embodiments, the methods provided herein further comprise post-amplification treatment of the nucleic acid molecules attached to the surface. For example, post-amplification treatment may comprise removing deoxyuridine primers after amplifying the nucleic acid molecules on the surface. Additional post-amplification treatment in the art may be used in preparation for sequencing.

In some embodiments, the methods provided herein further comprise washing the single-stranded nucleic acid molecules with a wash buffer prior to hybridizing sequencing primers to the single-stranded nucleic acid molecules. The washing may remove ethylene carbonate from the surface. In some embodiments, the wash buffer is a tris(hydroxymethyl)aminomethane (tris)-based wash buffer.

In some aspects, a method of preparing nucleic acid molecules for sequencing, comprises: attaching nucleic acid molecules to a surface; amplifying the nucleic acid molecules on the surface to generate double-stranded nucleic acid molecules; contacting the double-stranded nucleic acid molecules with ethylene carbonate to generate single-stranded nucleic acid molecules; washing the single-stranded nucleic acid molecules with a wash buffer; and, hybridizing sequencing primers to the single-stranded nucleic acid molecules, thereby generating sequencing hybrids.

In some aspects, a method of preparing nucleic acid molecules for sequencing, comprises: providing a surface comprising nucleic acid molecules immobilized thereto; amplifying the nucleic acid molecules on the surface to generate double-stranded nucleic acid molecules; contacting the double-stranded nucleic acid molecules with ethylene carbonate to generate single-stranded nucleic acid molecules; washing the single-stranded nucleic acid molecules with a wash buffer; and, hybridizing sequencing primers to the single-stranded nucleic acid molecules, thereby generating sequencing hybrids.

In another aspect, a method of sequencing nucleic acid molecules, comprises: attaching nucleic acid molecules to a surface; amplifying the nucleic acid molecules on the surface to generate double-stranded nucleic acid molecules; contacting the double-stranded nucleic acid molecules with ethylene carbonate to generate single-stranded nucleic acid molecules; washing the single-stranded nucleic acid molecules with a wash buffer; hybridizing sequencing primers to the single-stranded nucleic acid molecules, thereby generating sequencing hybrids; and, sequencing the single-stranded nucleic acid molecules using a plurality of sequencing flow steps, each sequencing flow step comprising combining the single-stranded nucleic acid molecules hybridized to the sequencing primers with non-terminating nucleotides, wherein at least a portion of the non-terminating nucleotides are labeled, and detecting the presence or absence of an incorporated non-terminating nucleotide.

### Definitions

As used herein, the singular forms “a,” “an,” and “the” include the plural reference unless the context clearly dictates otherwise.

Reference to “about” a value or parameter herein includes (and describes) variations that are directed to that value or parameter per se. For example, description referring to “about X” includes description of “X”.

The terms “individual,” “patient,” and “subject” are used synonymously, and refer to a mammal, and includes, but is not limited to, human, bovine, horse, feline, canine, rodent, or primate. In one embodiment, the subject is a human.

A “double-stranded nucleic acid molecule” refers to a nucleic acid molecule that includes at least one duplexed region. The double-stranded nucleic acid molecule need not be entirely duplexed, and may include regions of the nucleic acid molecule that are not duplexed.

A “single-stranded nucleic acid molecule” refers to a nucleic acid molecule that does not have any duplexed region.

A “flow order” refers to the order of separate nucleotide flows used to sequence a nucleic acid molecule using non-terminating nucleotides. The flow order may be divided into cycles of repeating units, and the flow order of the repeating units is termed a “flow-cycle order.” A “flow position” refers to the sequential position of a given separate nucleotide flow during the sequencing process.

A “non-terminating nucleotide” is a nucleic acid moiety that can be attached to a 3′ end of a polynucleotide using a polymerase or transcriptase, and that can have another non-terminating nucleic acid attached to it using a polymerase or transcriptase without the need to remove a protecting group or reversible terminator from the nucleotide. Naturally occurring nucleic acids are a type of non-terminating nucleic acid. Non-terminating nucleic acids may be labeled or unlabeled.

It is understood that aspects and variations of the invention described herein include “consisting of” and/or “consisting essentially of” aspects and variations.

When a range of values is provided, it is to be understood that each intervening value between the upper and lower limit of that range, and any other stated or intervening value in that states range, is encompassed within the scope of the present disclosure. Where the stated range includes upper or lower limits, ranges excluding either of those included limits are also included in the present disclosure.

The section headings used herein are for organization purposes only and are not to be construed as limiting the subject matter described. The description is presented to enable one of ordinary skill in the art to make and use the invention and is provided in the context of a patent application and its requirements. Various modifications to the described embodiments will be readily apparent to those persons skilled in the art and the generic principles herein may be applied to other embodiments. Thus, the present invention is not intended to be limited to the embodiment shown but is to be accorded the widest scope consistent with the principles and features described herein.

The figures illustrate processes according to various embodiments. In some examples, additional steps may be performed in combination with the exemplary processes. Accordingly, the operations as illustrated (and described in greater detail below) are exemplary by nature and, as such, should not be viewed as limiting.

The disclosures of all publications, patents, and patent applications referred to herein are each hereby incorporated by reference in their entireties. To the extent that any reference incorporated by reference conflicts with the instant disclosure, the instant disclosure shall control.

### Methods of Preparing Nucleic Acid Molecules for Sequencing on a Surface

Provided herein are methods of preparing nucleic acid molecules for sequencing on a surface. In some aspects, double-stranded nucleic acid molecules attached to the surface are contacted with ethylene carbonate to generate single-stranded nucleic acid molecules. The single-stranded nucleic acid molecules may be sequenced, for example, upon hybridization of sequencing primers to the single-stranded nucleic acid molecules, to generate sequencing data.

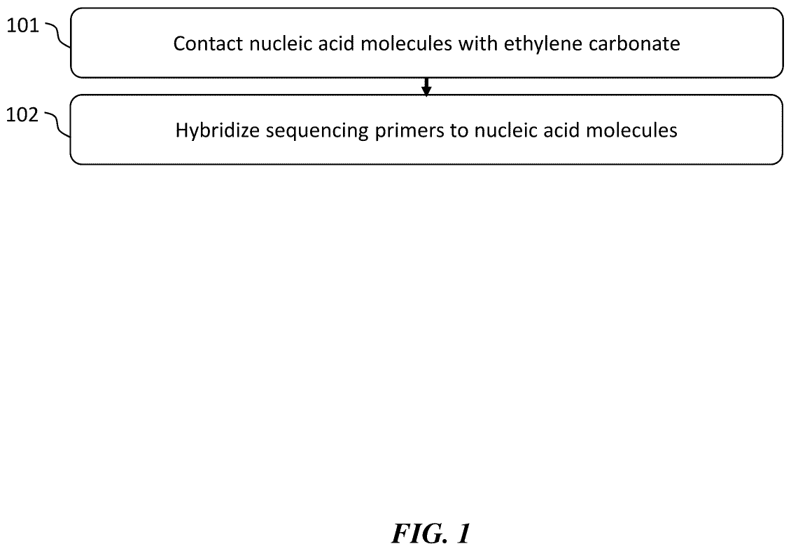

The methods described herein are compatible with next-generation nucleic acid molecule sequencing workflows. FIG. 1 shows an exemplary workflow for preparing nucleic acid molecules for sequencing on a surface, in accordance with some embodiments. Nucleic acid molecules (e.g., double-stranded nucleic acid molecules attached to a surface) may be contacted with ethylene carbonate 101 (e.g., generating single-stranded nucleic acid molecules attached to a surface). The nucleic acid molecules can be derived from a fluidic sample obtained from an individual. In some embodiments, the nucleic acid molecules comprise cell-free DNA molecules (e.g., cell free cDNA molecules). In some cases the surface may be a solid support such as a bead, which, in some embodiments, is immobilized to a wafer. In some cases, the surface may be the wafer. The nucleic acid molecules (e.g., single-stranded nucleic acid molecules) may then be hybridized with sequencing primers 102, thereby generating sequencing hybrids. In some cases, block 101 and 102 are performed in a single step (i.e., simultaneously). In such examples, the concentration of sequencing primer may be selected to favor the formation of sequencing hybrids (e.g., excess concentration of sequencing primer), and/or the sequencing primer may be designed to increase the stability of the sequencing hybrids (e.g., the sequencing primer comprises peptide nucleic acids (PNAs)).

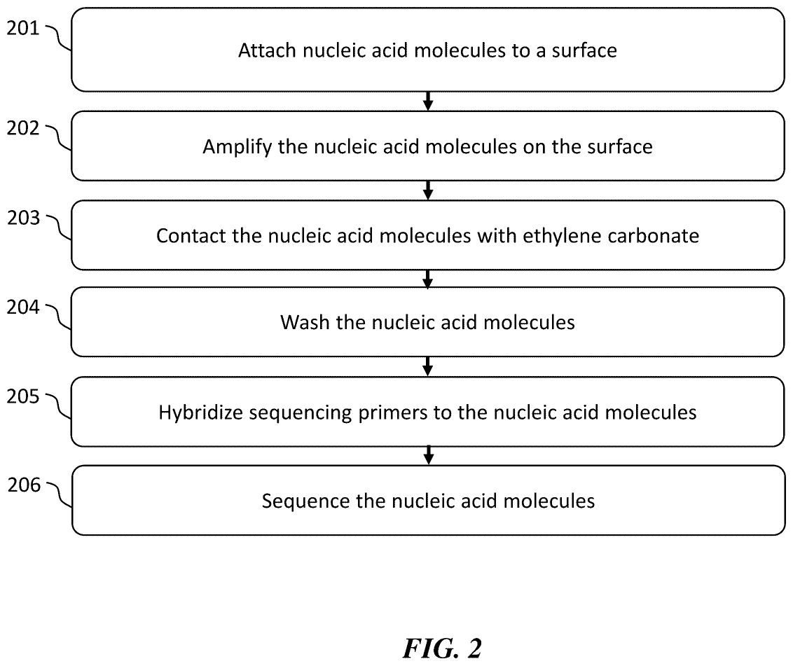

More specifically, methods of preparing nucleic acid molecules for next-generation sequencing provided herein are illustrated in FIG. 2. Nucleic acid molecules (e.g., nucleic acid molecules of a sequencing library) may be attached to a surface 201. Once attached to the surface, the nucleic acid molecules may be amplified on the surface 202. The amplification may be any one or more types of amplification that occur on a surface. For example, the amplification may be recombinase polymerase amplification (RPA) or molten recombinase polymerase amplification (mRPA), which do not require the thermocycling or the use of thermophilic enzymes. In another example, the amplification may be rolling circle amplification (RCA) and/or multiple displacement amplification (MDA). In some embodiments, the amplification is isothermal (e.g., conducted at a uniform temperature, for example within a deviation of 5° C., 4° C., 3° C., 2° C., 1° C. or less). In some cases, the amplification may be conducted at a temperature of about 43° C. Amplification of the nucleic acid molecules can generate double-stranded and/or at least partially double-stranded nucleic acid molecules attached to the surface. In some embodiments, the double-stranded nucleic acid molecules may be directly attached to the surface without amplification (e.g., the double-stranded nucleic acid molecules are not amplification products).

The double-stranded nucleic acid molecules attached to the surface may be contacted with ethylene carbonate 203, to generate single-stranded nucleic acid molecules attached to the surface. The single-stranded nucleic acid molecules may be washed with a wash buffer 204, to remove ethylene carbonate from the surface. In some embodiments, the washing 204 is not performed as part of the method of preparing nucleic acid molecules for next-generation sequencing. The single-stranded nucleic acid molecules may be hybridized with sequencing primers 205. In some embodiments, the sequencing primer is in excess concentration. In some embodiments, the sequencing primer is a peptide nucleic acid (PNA) primer. In some embodiments, the double-stranded nucleic acid molecules and/or the single-stranded nucleic acid molecules comprise a sequencing adaptor sequence. The sequencing adaptor sequence can comprise a sequencing primer hybridization sequence. In some embodiments, the sequencing primers may hybridize with the sequencing adaptor sequence, or a portion thereof, on the single-stranded nucleic acid molecules, thereby generating sequencing hybrids. In some embodiments, the contacting with ethylene carbonate 203 occurs in a single step with the hybridizing with sequencing primers. In some embodiments, the washing 204 is performed after the hybridization of sequencing primers 205. The single-stranded nucleic acid molecules may be sequenced, via the sequencing primers, thereby generating sequencing data 206. In some embodiments, the washing 204 is performed prior to the sequencing of the single-stranded nucleic acid molecules 206.

The use of ethylene carbonate to generate single-stranded nucleic acid molecules from double-stranded nucleic acid molecules on a surface allows for the combination of DNA denaturing (e.g., contacting with ethylene carbonate) and the hybridization of sequencing primers, into a compatible methodology. In some embodiments, the DNA denaturing with ethylene carbonate and hybridization of sequencing primers are performed in a single step (i.e., simultaneously). Furthermore, the denaturing step can be isothermal with upstream amplification and/or downstream sequencing analysis to result in a short, more streamlined, workflow. Thus, the provided methods of preparing nucleic acid molecules for sequencing represents an improvement over the current standard methods.

### Samples and Nucleic Acid Molecules

The nucleic acid molecules to be prepared for sequencing may be obtained from a fluidic sample, which may be obtained from an individual. In some embodiments, the individual is healthy. In some embodiments, the individual has, or is suspected of having, a disease (for example, a cancer). Fluidic samples are a relatively non-invasive method for obtaining a sample from an individual. Such fluidic samples can include, for example, a blood, plasma, saliva, fecal, or urine sample. Additionally, for residual, malignant, or other disease with no (or no significant) primary or solid diseased tissue, the fluidic sample allows one to obtain nucleic acid molecules associated with the diseased tissue without a tumor biopsy. The methods are therefore particularly useful when the location of the diseased tissue is unknown or the solid diseased tissue is too small to sample.

The fluidic sample taken from an individual may include cell-free DNA (or “cfDNA”). The cfDNA may include nucleic acid molecules derived from diseased tissue (e.g., cancer tissue) and nucleic acid molecules derived from the non-diseased tissue. The nucleic acid samples from which the sequencing data is obtained may be, but need not be, cfDNA. For example, a fluidic sample can provide other nucleic acids from which the sequencing data can be obtained. For example, if the disease is a blood disease (e.g., a hematological cancer), blood cells can be obtained from a blood sample, and the nucleic acid molecules from the blood cells can be sequenced to obtain the sequencing data. Thus, in some embodiments, the double-stranded nucleic acid molecules, and/or the single-stranded nucleic acid molecules generated therefrom, are DNA molecules. In some embodiments, the double-stranded nucleic acid molecules and/or the single-stranded nucleic acid molecules generated therefrom, are cDNA molecules.

### Surface

The nucleic acid molecules (e.g., double-stranded nucleic acid molecules and single-stranded nucleic acid molecules) may be covalently attached to a surface.

In some embodiments, a nucleic acid molecule may be attached to the surface through click chemistry. In some embodiments, the nucleic acid molecule is attached to the surface through amine-reactive crosslinker chemistry. In some embodiments, the nucleic acid molecule is attached to the surface through click chemistry and amine-reactive crosslinker chemistry. The nucleic acid molecule may comprise a click functional group, which may be directly conjugated with a click functional group of the surface. In some embodiments, the nucleic acid molecule comprises a 5′ click functional group. In some embodiments, the nucleic acid molecule comprises a 3′ click functional group. The click functional group of the nucleic acid molecule may react with a compatible click functional group of the surface, to attach the nucleic acid molecule to the surface.

In some implementations, an adaptor probe on the surface may be used to attach the nucleic acid molecules to the surface. The adaptor probe can be, for example, a nucleic acid adaptor probe. The adaptor probe may include a click functional group and a nucleic acid sequence that is complementary to, and is capable of hybridizing with, a nucleic acid molecule, or a portion thereof. In some embodiments, the adaptor probe hybridizes with the nucleic acid molecule, or a portion thereof. In some embodiments, the adaptor probe comprises a 5′ click functional group. In some embodiments, the adaptor probe comprises a 3′ click functional group. In some embodiments, the click functional group of the adaptor probe reacts with a compatible click functional group of the surface, to attach the nucleic acid molecule to the surface. In some embodiments, the adaptor probes are between about 15 and about 35 nucleic acids in length, such as between any of about 15 and about 30, between about 20 and about 35, or between about 18 and about 28 nucleotides in length. In some embodiments, the adaptor probes are greater than about 15 nucleotides in length, such as greater than any of about 20, 25, 30, 35, or more, nucleotides in length. In some embodiments, the adaptor probes are less than about 35 nucleic acids in length, such as less than any of about 30, 25, 20, 15, or fewer, nucleic acids in length.

In some embodiments, the nucleic acid molecules may be attached to the surface via their click functional groups using an enzyme-free click reaction (e.g., without use of a polymerase or a ligase for attachment). Example click chemistry for use with the methods described herein includes the click chemistry described in Gartner and Liu, The Generality of DNA-Templated Synthesis as a Basis for Evolving Non-Natural Small Molecules, Journal of the American Chemical Society, vol. 123, no. 28 (2001), pp. 6961-6963; Seckute et al., Rapid oligonucleotide-templated fluorogenic tetrazine ligations, Nucleic Acids Research, vol. 41, no. 15 (2013), pp. c148; and Patterson et al., Finding the right (bioorthogonal) chemistry, ACS Chemical Biololgy, vol. 9, no. 3 (2014), pp. 592-605, each of which is incorporated by reference herein in its entirety. In some embodiments, the click reaction is a template-independent reaction, for example as described in Xiong and Seela, Stepwise “Click” Chemistry for the Template Independent Construction of a Broad Variety of Cross-Linked Oligonucleotides: Influence of Linker Length, Position, and Linking Number on DNA Duplex Stability, Journal of Organic Chemistry, vol. 76, no. 14 (2011), pp. 5584-5597, which is incorporated by reference herein in its entirety). In some embodiments, the click reaction is a nucleophilic addition reaction. In some embodiments, the click reaction is a cyclopropane-tetrazine reaction. In some embodiments, the click reaction is a strain-promoted azide-alkyne cycloaddition (SPAAC) reaction. In some embodiments, the click reaction is an alkyne hydrothiolation reaction. In some embodiments, the click reaction is an alkene hydrothiolation reaction. In some embodiments, the click reaction is a strain-promoted alkyne-nitrone cycloaddition (SPANC) reaction. In some embodiments, the click reaction is an inverse electron-demand Diels-Alder (IED-DA) reaction. In some embodiments, the click reaction is a cyanobenzothiazole condensation reaction. In some embodiments, the click reaction is an aldehyde/ketone condensation reaction. In some embodiments, the click reaction is a Cu(I)-catalyzed azide-alkyne cycloaddition (CuAAC) reaction.

Various compatible click functional group pairs, capable of reacting with one another, are known in the art. For example, the compatible click functional group pairs may be, but are not limited to, azido/alkynyl, azido/cyclooctynyl, tetrazine/dienophile, thiol/alkynyl, cyano/1,2-amino thiol, and nitrone/cyclooctynyl. In some embodiments, adaptor probe click functional group and the surface click functional group are a cyclooctynyl and an azide, respectively. In some embodiments, adaptor probe click functional group and the surface click functional group are an azide and a cyclooctynyl, respectively. In some embodiments, adaptor probe click functional group and the surface click functional group are dibenzocyclooctyne (DBCO) and N₃, respectively. In some embodiments, adaptor probe click functional group and the surface click functional group are N₃ and DCBO, respectively.

In some embodiments, the surface may be a solid support, such as a bead. The bead may be a gel bead. In some embodiments, the surface may be attached to a wafer, wherein the wafer is a solid support. In some embodiments, the surface may be the wafer. The wafer may be a sequencing surface that is compatible with downstream sequencing analysis of the nucleic acid molecules on the surface. For example, sequencing (e.g., sequencing-by-synthesis) may be performed while the nucleic acid molecules, or derivatives thereof, are still immobilized on the wafer. In some embodiments, the nucleic acid sequencing data is obtained using surface-based sequencing of the nucleic acid molecules.

### Amplification

In some cases, the nucleic acid molecules (e.g., double-stranded nucleic acid molecules and/or single-stranded nucleic acid molecules) may not be amplified prior to attaching the nucleic acid molecules to a surface. In some cases, the nucleic acid molecules may be amplification products. In some cases, at least a portion of the nucleic acid molecules may be amplification products. In some cases, the nucleic acid molecules may be amplification products prior to attaching the nucleic acid molecules to the surface. For example, nucleic acid molecules may be attached to the surface, wherein a portion of the nucleic acid molecules are amplification products. The portion of the nucleic acid molecules attached to the surface that are not amplification products may be amplified following attachment to the surface.

Nucleic acid molecules in a sequencing library may be attached to the surface prior to the contacting with ethylene carbonate. A sequencing library may be generated by attaching adapter sequences to the 5′ and 3′ ends of sample sequences or template sequences (e.g., cDNA polynucleotides). The sequencing library may be attached to one or more surfaces (e.g., beads, wafers, etc.). In some cases, the sequencing library can be applied to a sequencing array surface containing DNA oligonucleotides attached to the surface. The DNA oligonucleotides can include sequences that hybridize to the adapter regions of the sequence library molecules. Amplification products that are double-stranded nucleic acid molecules can then be generated by amplification (e.g., on-surface amplification, which generates copies of the sequence library molecules and complements thereof).

The methods of preparing nucleic acid molecules for sequencing may comprise amplifying nucleic acid molecules of a sequencing library attached to the surface to generate amplified nucleic acid molecules. In some embodiments, the molecules of the sequencing library are double-stranded. In some embodiments, the molecules of the sequencing library are single-stranded. In some embodiments, the molecules of the sequencing library are amplified to generate nucleic acid amplification products (e.g., sequencing colonies) attached to a surface. For example, where the surface is a bead, after amplification, each bead may comprise its own sequencing colony attached thereto. In some cases, the beads are immobilized to a larger wafer surface. In some embodiments, the sequencing colonies comprise double-stranded nucleic acid molecules.

Nucleic acid molecules attached to the surface may be amplified by any one or more methods of amplification. Amplification of a nucleic acid molecule may be linear, exponential, or a combination thereof. Amplification may be surface based or non-surface based. Amplification may be emulsion based or non-emulsion based. Non-limiting examples of nucleic acid amplification methods include reverse transcription, primer extension, polymerase chain reaction (PCR), ligase chain reaction (LCR), helicase-dependent amplification, asymmetric amplification, rolling circle amplification (RCA), recombinase polymerase reaction (RPA), molten recombinase polymerase amplification (mRPA), loop mediated isothermal amplification (LAMP), nucleic acid sequence based amplification (NASBA), self-sustained sequence replication (3SR), and multiple displacement amplification (MDA). Amplification may be isothermal or non-isothermal. In some embodiments, the amplification may not require thermal melting or chemical melting of the template nucleic acid. In some embodiments, the amplification may not require the use of thermophilic enzymes.

The nucleic acid molecules attached to the surface may be amplified by isothermal amplifications. RPA and mRPA are example methods of isothermal amplification. RPA and mRPA are advantageous due to their simplicity, sensitivity, selectivity, compatibility with multiplexing, rapid amplification, as well as its operation at a low and constant temperature (e.g., isothermal), without the need for an initial denaturation step or the use of multiple primers. Furthermore, the enzymes used in mRPA may fold into a molten globule form, which can serve as a protected reaction pocket for amplification. Briefly, RPA and mRPA may comprise the following steps. First, a recombinase agent is contacted with a first and a second nucleic acid primer to form a first and a second nucleoprotein primer. In some embodiments, the primers comprise deoxyuridine (e.g., 2′-deoxyuridine-5′-triphosphate). The incorporation of deoxyuridine into the primers allows for a USER enzyme (a combination of Uracil DNA glycosylase (UDG) and the DNA glycosylase-lyase Endonuclease VIII) to create nicking sites in one of strands of the double-stranded target sequence, ultimately allowing improved detection (e.g., downstream sequencing analyses) and/or assist the signal generation of RPA assays.

Second, the first and second nucleoprotein primers are contacted to a double-stranded target sequence to form a first double-stranded structure at a first portion of said first strand and form a double-stranded structure at a second portion of said second strand so the 3′ ends of said first nucleic acid primer and said second nucleic acid primer are oriented towards each other on a given template DNA molecule. Third, the 3′ end of said first and second nucleoprotein primers are extended by DNA polymerases to generate first and second double-stranded nucleic acids, and first and second displaced strands of nucleic acid. Finally, the second and third steps are repeated until a desired degree of amplification is reached. The reaction may be incubated between 5 minutes and 16 hours, such as between 15 minutes and 3 hours or between 30 minutes and 2 hours. Additional description of RPA and mRPA can be found, for example, in U.S. Pat. No. 10,329,602B2 and WO2021/094746A1, each of which is herein incorporated by reference in its entirety.

In another example, RCA and/or MDA may be used to amplify the nucleic acid molecules. RCA may comprise the following operations. A template nucleic acid molecule (e.g., from a library) may be circularized to generate a circular template. In one example, a linear template molecule comprising a first adapter and a second adapter (e.g., at a first end and second end respectively, or at a single end) is circularized via splint ligation, in which a splint molecule is hybridized to the first adapter and the second adapter, and the ends of the template molecule are ligated. In another example, circularization may be performed without a splint. An amplification primer may be contacted and hybridized to the circular template, for example to at least a portion of a first and/or second adapter sequence, and extended using the circular template as a template, to generate a concatemer amplification product. The concatemer amplification product may comprise multiple units of (i) a sequence corresponding to the template insert sequence and (ii) a sequence configured to bind to, or corresponding to, a sequencing primer. In some cases, MDA may be performed with or subsequent to RCA. MDA may comprise the following operations. A plurality of primers may be contacted to a template concatemer, such as the concatemer amplification product of RCA, hybridized, and extended using the template concatemer as a template, to generate multiple concatemer strands. Each of the multiple concatemer strands may comprise units of (i) a sequence corresponding to the template insert sequence and (ii) a sequence configured to bind to, or corresponding to, a sequencing primer. In some cases, the primers can include both forward and reverse primers to generate concatemers in the forward and reverse directions.

In some cases, with or after amplification (e.g., RPA, mRPA, RCA, MDA, etc.), the amplicons may be enzymatically nicked. For example, a USER enzyme mix may be used for the cleavage reaction. The USER enzyme mix may comprise uracil DNA glycosylase (UDG), which removes the sugar and creates an abasic site (AP site), and endonuclease (e.g., endonuclease VIII), which binds to the AP Site and cleaves. In some cases, the enzyme mix may alternatively or additionally comprise APE1 enzyme. In some cases, the endonuclease may be replaced with an APEI enzyme in the USER enzyme mix.

In some embodiments, the nucleic acid molecules may not be amplified isothermally. In some embodiments, the nucleic acid molecules may be amplified isothermally. The isothermal amplification may occur at a similar temperature to downstream sequencing of the nucleic acid molecules. In some embodiments, the amplifying occurs between about 30° C. and about 50° C., such as between any of about 30° C. and about 40° C., between about 35° C. and about 45° C., or between about 40° C. and about 50° C. In some embodiments, the amplifying occurs at a temperature greater than about 30° C., such as greater than any of about 32° C., 34° C., 36° C., 38° C., 40° C., 42° C., 44° C., 46° C., 48° C., 50° C., or greater. In some embodiments, the amplifying occurs at a temperature less than about 50° C., such as less than any of about 48° C., 46° C., 44° C., 42° C., 40° C., 38° C., 36° C., 34° C., 32° C., 30° C., or less.

The amplifying may comprise the use of reagents. RPA and/or mRPA reagents can be lyophilized and in that form exhibit excellent stability at ambient temperatures for, at least, one year. In some embodiments, the reagents comprise a recombinase, a polymerase, a stabilizing agent (e.g., trehalose), and/or a crowding agent. A recombinase is an enzyme that can coat single-stranded DNA (ssDNA) to form filaments, which can then scan double-stranded DNA (dsDNA) for regions of sequence homology. Suitable recombinases include the E. coli RecA, RecO, or Rect protein, the T4-like bacteriophage uvsX protein, or any homologous protein or protein complex from any phyla. In some embodiments, recombinase concentrations may be, for example, between about 0.2-12 μM, 0.2-1 μM, 1-4 μM, 4-6 μM, and 6-12 μM. In some embodiments, the recombinase works in the presence of ATP, ATPγS, or other nucleoside triphosphates and their analogs.

The DNA polymerase may be a eukaryotic polymerase. In some embodiments, the eukaryotic polymerase includes, but is not limited to, pol-α, pol-β, pol-δ, pol-ε and derivatives and combinations thereof. In some embodiments, the DNA polymerase is a prokaryotic polymerase. Examples of prokaryotic polymerase include, but are not limited to, E. coli DNA polymerase I Klenow fragment, bacteriophage T4 gp43 DNA polymerase, Bacillus stearothermophilus polymerase I large fragment, Phi-29 DNA polymerase, T7 DNA polymerase, Bacillus subtilis Pol I, E. coli DNA polymerase I, E. coli DNA polymerase II, E. coli DNA polymerase III, E. coli DNA polymerase IV, E. coli DNA polymerase V and derivatives and combinations thereof. In some embodiments, the DNA polymerase is at a concentration of between 10,000 units/mL to 10 units/mL. In some embodiments, the DNA polymerase lacks 3′-5′ exonuclease activity. In some embodiments, the DNA polymerase contains strand-displacing properties.

The crowding agents used in the RPA and/or mRPA may include polyethylene glycol (PEG), dextran, and ficoll, or combinations and derivatives thereof. In some embodiments, the crowding agent is PEG1450, PEG3000, PEG8000, PEG10000, PEG compound molecular weight 15,000 to 20,000 (also known as Carbowax 20M), and a combination thereof.

In some embodiments, the amplification may further comprise use of a single-stranded DNA binding protein (SSB), or derivatives thereof. The single-stranded DNA binding protein may be the E. coli SSB or the T4 gp32 or a derivative or a combination of these proteins. gp32 derivatives may include, but are not limited to. gp32 (N), gp32 (C), gp32 (C) K3A, gp32 (C) R4Q, gp32 (C) R4T, gp32K3A, gp32R4Q, gp32R4T and a combination thereof. In some embodiments, the DNA binding protein is present at a concentration of between 1 μM and 30 μM.

In some embodiments, the amplification may further comprise use of accessory agents, such as but not limited to, magnesium acetate, betaine, trimethylamine N-oxide. In some embodiments, the order of reagent addition is important for the effectiveness of the amplification. For example, addition of magnesium acetate prior to the SSB may reduce spreading of amplicons, leading to decreased signal-to-noise ratio. The addition of trimethylamine N-oxide may likewise reduce spreading of amplicons. In some embodiments, the betaine can increase the amplification signal by enhancing the amplification of GC rich sequences. Furthermore, the synergy between reagents, such as magnesium acetate, betaine, and trimethylamine N-oxide can improve amplification quality overall.

Any of the proteins mentioned may also include use of its derivative. These proteins include, for example: recombinases, polymerase, SSBs, accessory agents, stabilizing agents (e.g., trehalose), and the like. In some embodiments, derivatives comprise protein fusions comprising sequence tags and/or protein tags, such as C terminus tag, N terminus tag, or C and N terminus tags. Appropriate tags may include, for example, 6-histidine, c-myc epitope, FLAG® octapeptide, Protein C, Tag-100, V5 epitope, VSV-G, Xpress, and hemagglutinin, β-galactosidase, thioredoxin, His-patch thioredoxin, IgG-binding domain, intein-chitin binding domain, T7 gene 10, glutathione-S-transferase (GST), green fluorescent protein (GFP), and maltose binding protein (MBP).

RCA and/or MDA reagents may comprise polymerases with strand displacement activity. Non-limiting examples of polymerases include DNA polymerases Bst, Bsm, and Vent without 5′-3′-exonuclease activity, phi29, T7 RNA polymerases, etc. Circularization reagents may comprise a ligase. Non-limiting examples include Taq DNA ligase, T3 DNA ligase, T4 DNA ligase, T7 DNA ligase, E. coli DNA ligase, TS2126 RNA ligase, Circligase™ ssDNA ligase, Thermophage™ ssDNA ligase, SplintR® ligase, etc.

The amplification can also include the addition of dNTPs. The dNTPs include, for example, dATP, dGTP, dCTP, and dTTP. In some embodiments, ATP, GTP, CTP, and UTP may also be included for synthesis of RNA primers. In embodiments, ddNTPs (ddATP, ddTTP, ddGTP and ddGTP) may be used to generate fragment ladders. In some embodiments, the dNTP is used at a concentration of between 1 μM to 200 μM of each NTP species, or be used as a mixture of dNTP and ddNTP.

Following amplification, e.g., RPA or mRPA, RCA, MDA, various post-amplification treatments may be performed to prepare the amplified nucleic acids for sequencing. For example, in some embodiments, deoxyuridine primers (e.g., 2′-deoxyuridine-5′-triphosphate) used during amplification are removed. Additional post-amplification treatments may be performed as necessary.

Amplification products that are treated by ethylene carbonate to generate single-stranded nucleic acid molecules, as described herein, may have any length. For example, the amplification products may be relatively shorter, with approximately less than 100 bp or less than 200 bp, or relatively longer, with approximately more than 200 bp. In some cases, an amplified nucleic acid molecule may have a length of at least about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400 bp or longer. Alternatively or in addition, an amplified nucleic acid molecule may have a length of at most about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400 bp or less.

### Ethylene Carbonate

A method of preparing nucleic acid molecules for sequencing as described herein comprises contacting double-stranded nucleic acid molecules attached to a surface with ethylene carbonate to generate single-stranded nucleic acid molecules. The ethylene carbonate can denature the double-stranded nucleic acid molecules to generate single-stranded nucleic acid molecules.

In some embodiments, the double-stranded nucleic acid molecules may be contacted with a solution comprising ethylene carbonate. The ethylene carbonate may be dissolved in water to form a solution comprising a specific concentration of ethylene carbonate. In some embodiments, the ethylene carbonate may be dissolved in a buffer. In some embodiments, the buffer may be a tris-based aqueous solution. In some embodiments, the ethylene carbonate provided to the double-stranded nucleic acid molecules has a concentration of between about 10% and about 50% volume/volume, such as between about any of 10% and 30% volume/volume, 35% and 45% volume/volume, or 40% and 50% volume/volume ethylene carbonate. In some embodiments, the ethylene carbonate provided to the double-stranded nucleic acid molecules has a concentration greater than about 10% volume/volume, such as greater than any of about 15%, 20% 25%, 30%, 35%, 40%, 45%, 50%, or greater, volume/volume ethylene carbonate. In some embodiments, the ethylene carbonate provided to the double-stranded nucleic acid molecules has a concentration less than about 50% volume/volume, such as less than any of about 45%, 40%, 35%, 30%, 25%, 20%, 15%, 10%, or less, volume/volume ethylene carbonate. In some embodiments, the ethylene carbonate provided to the double-stranded nucleic acid molecules has a concentration of 35% volume/volume.

In some embodiments, the contacting of the double-stranded nucleic acid molecules attached to a surface with ethylene carbonate may be implemented at a temperature of between about 35° C. and about 50° C., such as between about any of 35° C. and 40° C., 38° C. and 43° C., 40° C. and 48° C., or 45° C. and 50° C. In some embodiments, the double-stranded nucleic acid molecules may be contacted with the ethylene carbonate at a temperature greater than about 35° C., such as greater than any of about 38° C., 40° C., 42° C., 44° C., 46° C., 48° C., 50° C., or greater. In some embodiments, the double-stranded nucleic acid molecules may be contacted with the ethylene carbonate at a temperature less than about 50° C., such as greater than any of about 48° C., 46°° C., 44° C., 42° C., 40° C., 38° C., 35° C., or less. In some embodiments, the double-stranded nucleic acid molecules may be contacted with the ethylene carbonate at 43° C. In some embodiments, the double-stranded nucleic acid molecules may be contacted with the ethylene carbonate at room temperature. In some embodiments, where the double-stranded nucleic acids are amplification products (e.g., have been amplified using RPA, mRPA, RCA, or MDA), the contacting with ethylene carbonate can occur at a temperature that is substantially isothermal with the temperature at which the amplification occurs. In some embodiments, the contacting with ethylene carbonate can occur at a temperature that is substantially isothermal with the temperature at which the sequencing occurs. “Substantially isothermal” as used herein generally refers to a temperature that may minimally vary, such as vary by between about 1° C. and 5° C., from the temperature at which the amplification and/or sequencing occurs.

The duration of the contacting of the double-stranded nucleic acid molecules attached to a surface with ethylene carbonate can be adjusted or optimized, such that the contacting occurs for a period of time that is sufficient to generate single-stranded nucleic acid molecules from the nucleic acid molecules. In some embodiments, the contacting of the double-stranded nucleic acid molecules attached to a surface with ethylene carbonate can occur for between about 5 minutes and about 1 hour, such as between any of about 5 minutes and 30 minutes, 20 minutes and 40 minutes, 30 minutes and 50 minutes, and 40 and 60 minutes. In some embodiments, the double-stranded nucleic acid molecules may be contacted with ethylene carbonate for greater than about 5 minutes, such as greater than about any of 10 minutes, 15 minutes, 20 minutes, 25 minutes, 30 minutes, 35 minutes, 40 minutes, 45 minutes, 50 minutes, 55 minutes, 1 hour, or greater. In some embodiments, the double-stranded nucleic acid molecules may be contacted with ethylene carbonate for less than about 1 hour, such as greater than about any of 55 minutes, 50 minutes, 45 minutes, 40 minutes, 35 minutes, 30 minutes, 25 minutes, 20 minutes, 15 minutes, 10 minutes, 5 minutes, or less. In some embodiments, the double-stranded nucleic acid molecules may be contacted with ethylene carbonate for about 30 minutes.

### Washing

The methods provided herein may further comprise a washing step. In some embodiments, the single-stranded nucleic acid molecules are washed with a wash buffer prior to hybridizing sequencing primers to the single-stranded nucleic acid molecules. In some embodiments, the single-stranded nucleic acid molecules are washed with a wash buffer after hybridizing sequencing primers to the single-stranded nucleic acid molecules. In some embodiments, the single-stranded nucleic acid molecules are washed with a wash buffer prior to sequencing. The washing can remove residual ethylene carbonate from the surface prior to hybridizing sequencing primers and/or prior to sequencing the single-stranded nucleic acid molecules. In some embodiments, the washing clears the ethylene carbonate from the surface to allow for effective downstream sequencing. In some embodiments, the washing clear unhybridized sequencing primers from the surface to allow for effective downstream sequencing.

In some embodiments, the washing can comprise washing with a wash buffer. In some embodiments, the wash buffer is a tris (hydroxymethyl) aminomethane (Tris)-based buffer with a pH between about 7 and about 9. The wash buffer may comprise, but is not limited to, reagents such as Tris, ethylenediaminetetraacetic acid (EDTA), triton, and sodium dodecyl sulfate (SDS). In some embodiments, the washing can be repeated, such as repeated two, three, four, five, or more times. In some embodiments, the washing may be repeated a sufficient number of times to remove ethylene carbonate from the surface in preparation for the hybridization of sequencing primers. In some embodiments, the washing may be repeated a sufficient number of times to remove ethylene carbonate from the surface in preparation for sequencing of the single-stranded nucleic acid molecules. In some embodiments, the washing may be repeated a sufficient number of times to remove unhybridized sequencing primers from the surface in preparation for sequencing of the single-stranded nucleic acid molecules.

### Sequencing Primers

The methods of preparing nucleic acid molecules for sequencing can further comprise hybridizing sequencing primers to the single-stranded nucleic acid molecules generated from the double-stranded nucleic acid molecules attached to the surface. In some embodiments, the sequencing primers are nucleic acid primers. In some embodiments, the sequencing primers can comprise between about 5 and about 20 bases in length. In some embodiments, the sequencing primers can comprise about 12 bases in length. In some embodiments, the sequencing primer can comprise more than 20 bases in length. The nucleic acid primers may be used to amplify single-stranded nucleic acid molecules through amplification (e.g., PCR), to generate sequencing data associated with the single-stranded nucleic acid molecules.

The sequencing primers may be synthetic and/or modified nucleic acid primers comprising or consisting of synthetic or modified nucleotides. For example, the sequencing primers can comprise locked nucleic acid (LNA), peptide nucleic acid (PNA), and/or morpholino nucleotides. In some embodiments, the sequencing primers are PNA primers. The inclusion of synthetic and/or modified nucleic acids in the sequencing primer may increase the stability of the sequencing hybrids produced upon hybridization of the sequencing primers to single-stranded nucleic acid molecules. In some embodiments, the PNA sequencing primers have increase affinity for the single-stranded nucleic acid molecules compared to nucleic acid sequencing primers. A concentration of sequencing primers may be selected in order to facilitate hybridization of the sequencing primers to single-stranded nucleic acid molecules. For example, in cases where the DNA denaturing (e.g., contacting with ethylene carbonate) and hybridizing of sequencing primers are combined into a single step, the concentration of sequencing primers can be selected such that the hybridization reaction equilibrium favors the formation of sequencing hybrids. In some embodiments, the concentration of sequencing primers favors the formation of sequencing hybrids over the denaturing of the sequencing primers themselves. In some embodiments, the sequencing primers are in excess concentration (e.g., molar excess) compared to the single-stranded nucleic acid molecules. In some embodiments, the concentration of sequencing primers is any of about 2-fold excess, 5-fold excess, 10-fold excess, 20-fold excess, 50-fold excess, 100-fold excess, 200-fold excess, 500-fold excess, or more, compared to the single-stranded nucleic acid molecules.

The nucleic acid molecules can comprise a sequencing adaptor sequence. The sequencing adaptor sequence can comprise a sequencing primer hybridization sequence. The sequencing primer may hybridize with the sequencing primer hybridization sequence of the sequencing adaptor sequence on a single-stranded nucleic acid molecule. In some examples, one sequencing primer hybridizes with each sequencing primer hybridization sequence, or a portion thereof, on a single-stranded nucleic acid molecule.

Upon hybridization of the sequencing primers to the sequencing adaptor sequence of the single-stranded nucleic acid molecules, the sequencing primers can be used to sequence the single-stranded nucleic acid molecules, thereby generating sequencing data. Sequencing data may be generated, for example, by extending a sequencing primer hybridized with the single-stranded nucleic acid molecule, using a repeated flow-cycle order. The sequencing data may be representative of the extended sequencing primer strand, and sequencing information for the complementary template strand can be readily determined. A more detailed description of flow sequencing is provided herein.

### Sequencing

Nucleic acid molecules (e.g., single-stranded nucleic acid molecules prepared according to the methods provided herein) may be sequenced using any suitable sequencing method to obtain sequencing data from the nucleic acid molecules. In some embodiments, the nucleic acid molecules may comprise a sequencing adaptor sequence, wherein the sequencing adaptor sequence comprises a sequencing primer hybridization sequence. The sequencing primer may hybridize with the sequencing primer hybridization sequence of the sequencing adaptor sequence on the nucleic acid molecule, and can be used to sequence the nucleic acid molecule, thus generating sequencing data.

Exemplary sequencing methods can include, but are not limited to, high-throughput sequencing, next-generation sequencing, sequencing-by-synthesis, flow sequencing, massively-parallel sequencing, shotgun sequencing, single-molecule sequencing, nanopore sequencing, pyrosequencing, semiconductor sequencing, sequencing-by-ligation, sequencing-by-hybridization, RNA-Seq, digital gene expression, single molecule sequencing by synthesis (SMSS), clonal single molecule array, sequencing by ligation, and Maxim-Gilbert sequencing. In some embodiments, the nucleic acid molecules may be sequenced using a high-throughput sequencer, such as an Illumina HiSeq2500, Illumina HiSeq3000, Illumina HiSeq4000, Illumina HiSeqX, Roche 454, Life Technologies Ion Proton, or open sequencing platform as described in U.S. Pat. No. 10,267,790, which is incorporated herein by reference in its entirety.

Other methods of sequencing and sequencing systems are known in the art. In some embodiments, the nucleic acid molecules are sequenced using a sequencing-by synthesis (SBS) method. In some embodiments, the nucleic acid molecules are sequenced using a “natural sequencing-by-synthesis” or “non-terminated sequencing-by-synthesis” method (see, U.S. Pat. No. 8,772,473, which is incorporated herein by reference in its entirety).

### Flow Sequencing Methods

Sequencing data associated with the single-stranded nucleic acid molecules prepared according to the methods provided herein can be generated using a flow sequencing method that includes extending a primer bound to a template polynucleotide molecule according to a pre-determined flow cycle where, in any given flow position, a single base type of nucleotide is accessible to the extending primer. In some embodiments, the single-stranded nucleic acid molecules may be sequenced using a plurality of sequencing flow steps, each sequencing flow step comprising contacting the single-stranded nucleic acid molecules hybridized to the sequencing primers with nucleotides, wherein at least a portion of the nucleotides are labeled, and detecting the presence or absence of an incorporated nucleotide. In some embodiments, at least some of the nucleotides of the particular type include a label, which upon incorporation of the labeled nucleotides into the extending primer renders a detectable signal. In some embodiments, the at least a portion of the nucleotides is less than all of the nucleotides in each sequencing flow step. The resulting sequence by which such nucleotides are incorporated into the extended primer are expected to be the reverse complement of the sequence of the template polynucleotide molecule. In some embodiments, for example, sequencing data may be generated using a flow sequencing method that includes extending a primer using labeled nucleotides, and detecting the presence or absence of a labeled nucleotide incorporated into the extending primer. While the following description is provided in reference to flow sequencing methods, it is understood that other sequencing methods may be used to sequence all or a portion of the sequenced region.

Flow sequencing includes the use of nucleotides to extend the primer hybridized to the polynucleotide. Nucleotides of a given base type (e.g., A, C, G, T, U, etc.) can be mixed with hybridized templates to extend the primer if a complementary base is present in the template strand. In some embodiments, the nucleotides in each sequencing flow step comprise nucleotides of a same base type. The nucleotides may be, for example, non-terminating nucleotides. When the nucleotides are non-terminating, more than one consecutive base can be incorporated into the extending primer strand if more than one consecutive complementary base is present in the template strand. The non-terminating nucleotides contrast with nucleotides having 3′ reversible terminators, wherein a blocking group is generally removed before a successive nucleotide is attached. If no complementary base is present in the template strand, primer extension ceases until a nucleotide that is complementary to the next base in the template strand is introduced. At least a portion of the nucleotides can be labeled so that incorporation can be detected. Most commonly, only a single nucleotide type is introduced at a time (i.e., discretely added), although two or three different types (e.g., base type) of nucleotides may be simultaneously introduced in certain embodiments. This methodology can be contrasted with sequencing methods that use a reversible terminator, wherein primer extension is stopped after extension of every single base before the terminator is reversed to allow incorporation of the next succeeding base.

The nucleotides can be introduced at a flow order during the course of primer extension, which may be further divided into flow cycles. The flow cycles are a repeated order of nucleotide flows, and may be of any length. Nucleotides are added stepwise, which allows incorporation of the added nucleotide to the end of the sequencing primer of a complementary base in the template strand is present. Solely by way of example, the flow order of a flow cycle may be A-T-G-C, or the flow cycle order may be A-T-C-G. Alternative orders may be readily contemplated by one skilled in the art. The flow cycle order may be of any length, although flow cycles containing four unique base type (A, T, C, and G in any order) are most common. In some embodiments, the flow cycle includes 5, 6, 7, 8, 9, 10, 11, 12, 13, 14, 15, 16, 17, 18, 19, 20 or more separate nucleotide flows in the flow cycle order. Solely by way of example, the flow cycle order may be T-C-A-C-G-A-T-G-C-A-T-G-C-T-A-G, with these 16 separately provided nucleotides provided in this flow-cycle order for several cycles. Between the introductions of different nucleotides, unincorporated nucleotides may be removed, for example by washing the sequencing platform with a wash fluid.

A polymerase can be used to extend a sequencing primer by incorporating one or more nucleotides at the end of the primer in a template-dependent manner. In some embodiments, the polymerase is a DNA polymerase. The polymerase may be a naturally occurring polymerase or a synthetic (e.g., mutant) polymerase. The polymerase can be added at an initial step of primer extension, although supplemental polymerase may optionally be added during sequencing, for example with the stepwise addition of nucleotides or after a number of flow cycles. Exemplary polymerases include a DNA polymerase, an RNA polymerase, a thermostable polymerase, a wild-type polymerase, a modified polymerase, Bst DNA polymerase, Bst 2.0 DNA polymerase Bst 3.0 DNA polymerase, Bsu DNA polymerase, E. coli DNA polymerase I, T7 DNA polymerase, bacteriophage T4 DNA polymerase F29 (phi29) DNA polymerase, Taq polymerase, Tth polymerase, Tli polymerase, Pfu polymerase, and SeqAmp DNA polymerase.

The introduced nucleotides can include labeled nucleotides when determining the sequence of the template strand, and the presence or absence of an incorporated labeled nucleic acid can be detected to determine a sequence. The label may be, for example, an optically active label (e.g., a fluorescent label) or a radioactive label, and a signal emitted by or altered by the label can be detected using a detector. The presence or absence of a labeled nucleotide incorporated into a primer hybridized to a template polynucleotide can be detected, which allows for the determination of the sequence (for example, by generating a flowgram). In some embodiments, the labeled nucleotides are labeled with a fluorescent, luminescent, or other light-emitting moiety. In some embodiments, the label is attached to the nucleotide via a linker. In some embodiments, the linker is cleavable, e.g., through a photochemical or chemical cleavage reaction. For example, the label may be cleaved after detection and before incorporation of the successive nucleotide(s). In some embodiments, the label (or linker) is attached to the nucleotide base, or to another site on the nucleotide that does not interfere with elongation of the nascent strand of DNA. In some embodiments, the linker comprises a disulfide or PEG-containing moiety.

In some embodiments, the nucleotides introduced include only unlabeled nucleotides, and in some embodiments the nucleotides include a mixture of labeled and unlabeled nucleotides. For example, in some embodiments, the portion of labeled nucleotides compared to total nucleotides is about 90% or less, about 80% or less, about 70% or less, about 60% or less, about 50% or less, about 40% or less, about 30% or less, about 20% or less, about 10% or less, about 5% or less, about 4% or less, about 3% or less, about 2.5% or less, about 2% or less, about 1.5% or less, about 1% or less, about 0.5% or less, about 0.25% or less, about 0.1% or less, about 0.05% or less, about 0.025% or less, or about 0.01% or less. In some embodiments, the portion of labeled nucleotides compared to total nucleotides is about 100%, about 95% or more, about 90% or more, about 80% or more about 70% or more, about 60% or more, about 50% or more, about 40% or more, about 30% or more, about 20% or more, about 10% or more, about 5% or more, about 4% or more, about 3% or more, about 2.5% or more, about 2% or more, about 1.5% or more, about 1% or more, about 0.5% or more, about 0.25% or more, about 0.1% or more, about 0.05% or more, about 0.025% or more, or about 0.01% or more. In some embodiments, the portion of labeled nucleotides compared to total nucleotides is about 0.01% to about 100%, such as about 0.01% to about 0.025%, about 0.025% to about 0.05%, about 0.05% to about 0.1%, about 0.1% to about 0.25%, about 0.25% to about 0.5%, about 0.5% to about 1%, about 1% to about 1.5%, about 1.5% to about 2%, about 2% to about 2.5%, about 2.5% to about 3%, about 3% to about 4%, about 4% to about 5%, about 5% to about 10%, about 10% to about 20%, about 20% to about 30%, about 30% to about 40%, about 40% to about 50%, about 50% to about 60%, about 60% to about 70%, about 70% to about 80%, about 80% to about 90%, about 90% to less than 100%, or about 90% to about 100%.

Prior to generating the sequencing data, the polynucleotide is hybridized to a sequencing primer to generate a hybridized template. The polynucleotide may be ligated to an adapter during sequencing library preparation. The adapter can include a hybridization sequence that hybridizes to the sequencing primer. For example, the hybridization sequence of the adapter may be a uniform sequence across a plurality of different polynucleotides, and the sequencing primer may be a uniform sequencing primer. This allows for multiplexed sequencing of different polynucleotides in a sequencing library.

The polynucleotide may be attached to a surface (such as a solid support) for sequencing. The polynucleotides may be amplified (for example, by bridge amplification or other amplification techniques) to generate polynucleotide sequencing colonies. The amplified polynucleotides within the cluster are substantially identical or complementary (some errors may be introduced during the amplification process such that a portion of the polynucleotides may not necessarily be identical to the original polynucleotide). Colony formation allows for signal amplification so that the detector can accurately detect incorporation of labeled nucleotides for each colony. In some cases, the colony is formed on a bead using emulsion PCR and the beads are distributed over a sequencing surface. Examples for systems and methods for sequencing can be found in U.S. patent Ser. No. 11/118,223, which is incorporated herein by reference in its entirety.

The primer hybridized to the polynucleotide is extended through the nucleic acid molecule using the separate nucleotide flows according to the flow order (which may be cyclical according to a flow-cycle order), and incorporation of a nucleotide can be detected as described above, thereby generating the sequencing data set for the nucleic acid molecule.

Primer extension using flow sequencing allows for long-range sequencing on the order of hundreds or even thousands of bases in length. The number of flow steps or cycles can be increased or decreased to obtain the desired sequencing length. Extension of the primer can include one or more flow steps for stepwise extension of the primer using nucleotides having one or more different base types. In some embodiments, extension of the primer includes between 1 and about 1000 flow steps, such as between 1 and about 10 flow steps, between about 10 and about 20 flow steps, between about 20 and about 50 flow steps, between about 50 and about 100 flow steps, between about 100 and about 250 flow steps, between about 250 and about 500 flow steps, or between about 500 and about 1000 flow steps. The flow steps may be segmented into identical or different flow cycles. The number of bases incorporated into the primer depends on the sequence of the sequenced region, and the flow order used to extend the primer. In some embodiments, the sequenced region is about 1 base to about 4000 bases in length, such as about 1 base to about 10 bases in length, about 10 bases to about 20 bases in length, about 20 bases to about 50 bases in length, about 50 bases to about 100 bases in length, about 100 bases to about 250 bases in length, about 250 bases to about 500 bases in length, about 500 bases to about 1000 bases in length, about 1000 bases to about 2000 bases in length, or about 2000 bases to about 4000 bases in length.

### Sequencing Data Sets

Sequencing data can be generated based on the detection of an incorporated nucleotide and the order of nucleotide introduction. Take, for example, the flowing extended sequences (i.e., each reverse complement of a corresponding template sequence): CTG, CAG, CCG, CGT, and CAT (assuming no preceding sequence or subsequent sequence subjected to the sequencing method), and a repeating flow cycle of T-A-C-G (that is, sequential addition of T, A, C, and G nucleotides in repeating cycles). A particular type of nucleotides at a given flow position would be incorporated into the primer only if a complementary base is present in the template polynucleotide. An exemplary resulting flowgram is shown in Table 1, where 1 indicates incorporation of an introduced nucleotide and 0 indicates no incorporation of an introduced nucleotide. The flowgram can be used to derive the sequence of the template strand. For example, the sequencing data (e.g., flowgram) discussed herein represent the sequence of the extended primer strand, and the reverse complement of which can readily be determined to represent the sequence of the template strand. An asterisk (*) in Table 1 indicates that a signal may be present in the sequencing data if additional nucleotides are incorporated in the extended sequencing strand (e.g., a longer template strand).

TABLE 1
Exemplary Sequencing Data.

| | Cycle 1 | | | | Cycle 2 | | | | Cycle 3 | | | |
|---|---|---|---|---|---|---|---|---|---|---|---|---|
| Flow Position | 1 | 2 | 3 | 4 | 5 | 6 | 7 | 8 | 9 | 10 | 11 | 12 |
| Base in Flow | T | A | C | G | T | A | C | G | T | A | C | G |
| Extended | 0 | 0 | 1 | 0 | 1 | 0 | 0 | 1 | * | * | * | * |
| sequence: CTG | | | | | | | | | | | | |
| Extended | 0 | 0 | 1 | 0 | 0 | 1 | 0 | 1 | * | * | * | * |
| sequence: CAG | | | | | | | | | | | | |
| Extended | 0 | 0 | 2 | 1 | * | * | * | * | * | * | * | * |
| sequence: CCG | | | | | | | | | | | | |
| Extended | 0 | 0 | 1 | 1 | 1 | * | * | * | * | * | * | * |
| sequence: CGT | | | | | | | | | | | | |
| Extended | 0 | 0 | 1 | 0 | 0 | 1 | 0 | 0 | 1 | * | * | * |
| sequence: CAT | | | | | | | | | | | | |

The flowgram may be binary or non-binary. A binary flowgram detects the presence (1) or absence (0) of an incorporated nucleotide. A non-binary flowgram can more quantitatively determine a number of incorporated nucleotides from each stepwise introduction. For example, an extended sequence of CCG would include incorporation of two C bases in the extending primer within the same C flow (e.g., at flow position 3), and signals emitted by the labeled base would have an intensity greater than an intensity level corresponding to a single base incorporation. This is shown in Table 1. The non-binary flowgram also indicates the presence or absence of the base, and can provide additional information including the number of bases likely incorporated into each extending primer at the given flow position. The values do not need to be integers. In some cases, the values can be reflective of uncertainty and/or probabilities of a number of bases being incorporated at a given flow position.

In some embodiments, the sequencing data set includes flow signals representing a base count indicative of the number of bases in the sequenced nucleic acid molecule that are incorporated at each flow position. For example, as shown in Table 1, the primer extended with a CTG sequence using a T-A-C-G flow cycle order has a value of 1 at position 3, indicating a base count of 1 at that position (the 1 base being C, which is complementary to a G in the sequenced template strand). Also in Table 1, the primer extended with a CCG sequence using the T-A-C-G flow cycle order has a value of 2 at position 3, indicating a base count of 2 at that position for the extending primer during this flow position. Here, the 2 bases refer to the C-C sequence at the start of the CCG sequence in the extending primer sequence, and which is complementary to a G-G sequence in the template strand.

The flow signals in the sequencing data set may include one or more statistical parameters indicative of a likelihood or confidence interval for one or more base counts at each flow position. In some embodiments, the flow signal is determined from an analog signal that is detected during the sequencing process, such as a fluorescent signal of the one or more bases incorporated into the sequencing primer during sequencing. In some cases, the analog signal can be processed to generate the statistical parameter. For example, a machine-learning algorithm can be used to correct for context effects of the analog sequencing signal as described in published International patent application WO 2019084158 A1, which is incorporated by reference herein in its entirety. Although an integer number of zero or more bases are incorporated at any given flow position, a given analog signal many not perfectly match with the analog signal. Therefore, given the detected signal, a statistical parameter indicative of the likelihood of a number of bases incorporated at the flow position can be determined. Solely by way of example, for the CCG sequence in Table 1, the likelihood that the flow signal indicates 2 bases incorporated at flow position 3 may be 0.999, and the likelihood that the flow signal indicates 1 base incorporated at flow position 3 may be 0.001. The sequencing data set may be formatted as a sparse matrix, with a flow signal including a statistical parameter indicative of a likelihood for a plurality of base counts at each flow position.

Additional details regarding exemplary flow sequencing methods for use with the methods described herein includes the flow sequencing described in US 2020/0392584 A1, US 2020/0377937 A1 and US 2020/0372971 A1, each of which is incorporated herein by reference in its entirety.

### EXEMPLARY EMBODIMENTS

The following embodiments are exemplary and are not intended to limit the scope of the invention described herein.

Embodiment 1. A method of preparing nucleic acid molecules for sequencing, comprising:
contacting double-stranded nucleic acid molecules attached to a surface with ethylene carbonate to generate single-stranded nucleic acid molecules attached to the surface; and,
hybridizing sequencing primers to the single-stranded nucleic acid molecules, thereby generating sequencing hybrids.

Embodiment 2. The method of embodiment 1, wherein the ethylene carbonate has a concentration of between about 10% and about 50% volume/volume.

Embodiment 3. The method of embodiments 1 or 2, wherein the contacting is implemented at a temperature of between about 35° C. and about 50° C.

Embodiment 4. The method of any one of embodiments 1-3, wherein the double-stranded nucleic acid molecules attached to the surface are contacted with the ethylene carbonate for about 5 minutes or more.

Embodiment 5. The method of any one of embodiments 1-4, wherein the sequencing primer is a nucleic acid primer.

Embodiment 6. The method of any one of embodiments 1-4, wherein the sequencing primer is a peptide nucleic acid (PNA) primer.

Embodiment 7. The method of embodiment 6, wherein the PNA primer has increased hybridization affinity with the single-strand nucleic acid molecules compared to a nucleic acid primer.

Embodiment 8. The method of any one of embodiments 1-7, wherein the sequencing primer concentration is selected so that the hybridization reaction equilibrium favors the formation of sequencing hybrids.

Embodiment 9. The method of embodiment 8, wherein the sequencing primers are in excess concentration compared to the single-stranded nucleic acid molecules.

Embodiment 10. The method of any one of embodiments 1-9, wherein the contacting and hybridizing occur simultaneously.

Embodiment 11. The method of any one of embodiments 1-10, wherein the double-stranded nucleic acid molecules or the single-stranded nucleic acid molecules are derived from a fluidic sample obtained from an individual.

Embodiment 12. The method of embodiment 11, wherein the fluidic sample is a blood sample, a plasma sample, a saliva sample, a urine sample, or a fecal sample.

Embodiment 13. The method of embodiment 10 or 11, wherein the fluidic sample comprises cell-free nucleic acid molecules.

Embodiment 14. The method of any one of embodiments 10-13, wherein the fluidic sample comprises DNA molecules.

Embodiment 15. The method of any one of embodiments 10-14, wherein the fluidic sample comprises cDNA molecules.

Embodiment 16. The method of any one of embodiments 1-15, wherein the double-stranded nucleic acid molecules or the single-stranded nucleic acid molecules comprise a sequencing adaptor sequence, wherein the sequencing adaptor sequence comprises a sequencing primer hybridization sequence.

Embodiment 17. The method of any one of embodiments 1-16, wherein the double-stranded nucleic acid molecules or the single-stranded nucleic acid molecules are amplification products.

Embodiment 18. The method of any one of embodiments 1-17, wherein the double-stranded nucleic acid molecules or the single-stranded nucleic acid molecules are covalently attached to the surface.

Embodiment 19. The method of any one of embodiments 1-18, wherein the double-stranded nucleic acid molecules or the single-stranded nucleic acid molecules are attached to the surface using click chemistry or amine-reactive crosslinker chemistry.

Embodiment 20. The method of any one of embodiments 1-19, wherein the surface is a bead.

Embodiment 21. The method of embodiment 20, wherein the bead is a gel bead.

Embodiment 22. The method of any one of embodiments 1-21, wherein the surface is immobilized to a wafer.

Embodiment 23. The method of any one of embodiments 1-22, further

comprising attaching nucleic acid molecules in a sequencing library to the surface prior to the contacting with ethylene carbonate.

Embodiment 24. The method of embodiment 23, further comprising amplifying the nucleic acid molecules in the sequencing library attached to the surface prior to the contacting with ethylene carbonate, thereby generating sequencing colonies comprising the double-stranded nucleic acid molecules attached to the surface.

Embodiment 25. The method of embodiment 24, wherein the nucleic acid molecules are amplified isothermally.

Embodiment 26. The method of embodiment 25, wherein the amplifying occurs between about 30° C. and about 50° C.

Embodiment 27. The method of any one of embodiments 24-26, wherein the amplifying comprises one or more of rolling circle amplification (RCA), multiple displacement amplification (MDA), recombinase polymerase amplification (RPA), and molten recombinase polymerase amplification (mRPA).

Embodiment 28. The method of embodiment 27, wherein the amplifying comprises use of reagents selected from the group consisting of polymerases, recombinases, single-stranded DNA binding proteins, magnesium acetate, betaine, formamide, tetramethyl ammonium chloride, sodium dodecyl sulfate (SDS), and trimethylamine N-oxide.

Embodiment 29. The method of any one of embodiments 24-28, further comprising removing deoxyuridine primers after amplifying the nucleic acid molecules on the surface.

Embodiment 30. The method of any one of embodiments 1-29, further comprising washing the single-stranded nucleic acid molecules with a wash buffer prior to the hybridizing of sequencing primers to the single-stranded nucleic acid molecules.

Embodiment 31. The method of any one of embodiments 1-30, further comprising washing the sequencing hybrids with a wash buffer.

Embodiment 32. The method of embodiment 30 or 31, wherein the washing is repeated two or more times.

Embodiment 33. The method of any one of embodiments 30-32, wherein the wash buffer comprises tris (hydroxymethyl) aminomethane (tris), ethylenediaminetetraacetic acid (EDTA), triton, or sodium dodecyl sulfate (SDS).

Embodiment 34. The method of any one of embodiments 1-32, further comprising sequencing the single-stranded nucleic acid molecules, thereby generating sequencing data.

Embodiment 35. The method of embodiment 34, wherein the single-stranded nucleic acid molecules are sequenced using a plurality of sequencing flow steps, each sequencing flow step comprising contacting the sequencing hybrids with nucleotides, wherein at least a portion of the nucleotides are labeled, and detecting the presence or absence of an incorporated nucleotide.

Embodiment 36. The method of embodiment 35, wherein the nucleotides in each sequencing flow step comprise nucleotides of a same base type.

Embodiment 37. The method of embodiments 35 or 36, wherein the at least a portion of the nucleotides is less than all of the nucleotides in each sequencing flow step.

Embodiment 38. The method of any one of embodiments 35-37, wherein the nucleotides are non-terminating nucleotides.

Embodiment 39. The method of any one of embodiments 35-38, wherein the sequencing data comprises flow signals at the plurality of sequencing flow steps.

Embodiment 40. The method of embodiment 39, wherein the flow signals are used to determine a base count indicative of a number of bases sequenced at each flow step.

Embodiment 41. The method of embodiments 39 or 40, wherein the flow signals are used to determine a statistical parameter indicative of a likelihood for at least one base count at each flow step, wherein the base count is indicative of a number of bases of a given single-stranded nucleic acid molecule sequenced at a given flow step.

Embodiment 42. A method of preparing nucleic acid molecules for sequencing, comprising:
providing nucleic acid molecules attached to a surface;
amplifying the nucleic acid molecules on the surface to generate double-stranded nucleic acid molecules;
contacting the double-stranded nucleic acid molecules with ethylene carbonate to generate single-stranded nucleic acid molecules;
washing the single-stranded nucleic acid molecules with a wash buffer; and,
hybridizing sequencing primers to the single-stranded nucleic acid molecules, thereby generating sequencing hybrids.

Embodiment 43. The method of embodiment 42, wherein the amplifying is isothermal.

Embodiment 44. The method of embodiment 42 or 43, wherein the amplifying comprises one or more of rolling circle amplification (RCA), multiple displacement amplification (MDA), recombinase polymerase amplification (RPA), and molten recombinase polymerase amplification (mRPA).

Embodiment 45. A method of sequencing nucleic acid molecules, comprising: providing nucleic acid molecules attached to a surface;
amplifying the nucleic acid molecules on the surface to generate double-stranded nucleic acid molecules;
contacting the double-stranded nucleic acid molecules with ethylene carbonate to generate single-stranded nucleic acid molecules;
washing the single-stranded nucleic acid molecules with a wash buffer;
hybridizing sequencing primers to the single-stranded nucleic acid molecules, thereby generating sequencing hybrids; and,
sequencing the single-stranded nucleic acid molecules using a plurality of sequencing flow steps, each sequencing flow step comprising contacting the sequencing hybrids with non-terminating nucleotides, wherein at least a portion of the non-terminating nucleotides are labeled, and detecting the presence or absence of an incorporated non-terminating nucleotide.

Embodiment 46. The method of embodiment 45, wherein the amplifying is isothermal.

Embodiment 47. The method of embodiments 45 or 46, wherein the amplifying comprises one or more of rolling circle amplification (RCA), multiple displacement amplification (MDA), recombinase polymerase amplification (RPA), and molten recombinase polymerase amplification (mRPA).

Embodiment 48. The method of any one of embodiments 45-47, further comprising generating sequencing data, wherein the sequencing data comprises flow signals detected at the plurality of sequencing flow steps.

Embodiment 49. The method of any one of embodiments 45-48, wherein the flow signals are used to determine a base count indicative of a number of bases sequenced at each flow step.

Embodiment 50. The method of embodiments 48 or 49, wherein the flow signals are used to determine a statistical parameter indicative of a likelihood for at least one base count at each flow step, wherein the base count is indicative of a number of bases of a given single-stranded nucleic acid molecule sequenced at a given flow step.

### EXAMPLES

The application may be better understood by reference to the following non-limiting examples, which are provided as exemplary embodiments of the application. The following examples are presented in order to more fully illustrate embodiments and should in no way be construed as limiting the scope of the application. While certain embodiments of the present application have been shown and described herein, it will be obvious that such embodiments are provided by way of example only. Numerous variations, changes, and substitutions may occur to those skilled in the art without departing from the spirit and scope of the invention. It should be understood that various alternatives to the embodiments described herein may be employed in practicing the methods described herein.

### Example 1: Preparing Nucleic Acid Molecules for Sequencing

This example illustrates a method for denaturing double-stranded nucleic acid molecules in preparation for sequencing on a sequencing surface (e.g., a wafer).

Sample cells were lysed using a lysis buffer, and liquid lysate separated using centrifugation. The separated liquid lysate was incubated with beads that are further attached to a sequencing surface (e.g., a wafer). Nucleic acid molecules of interest in the liquid lysate were isolated using the beads by separating and washing the beads. The nucleic acid molecules were amplified isothermally using recombinase polymerase amplification (RPA) or molten recombinase polymerase amplification (mRPA) on the wafer to generate double-stranded nucleic acid molecules. The double-stranded nucleic acid molecules were then contacted with 35% ethylene carbonate or 100 mM sodium hydroxide (NaOH) to generate single-stranded nucleic acid molecules, as shown in Table 2.

TABLE 2
Exemplary denaturing conditions.

| | | | Time |
|---|---|---|---|
| Chemical | Concentration | Temperature | (minutes) |
| NaOH | 100 mM | Room temperature | 2 |
| Ethylene carbonate | 35% | 43° C. | 30 |

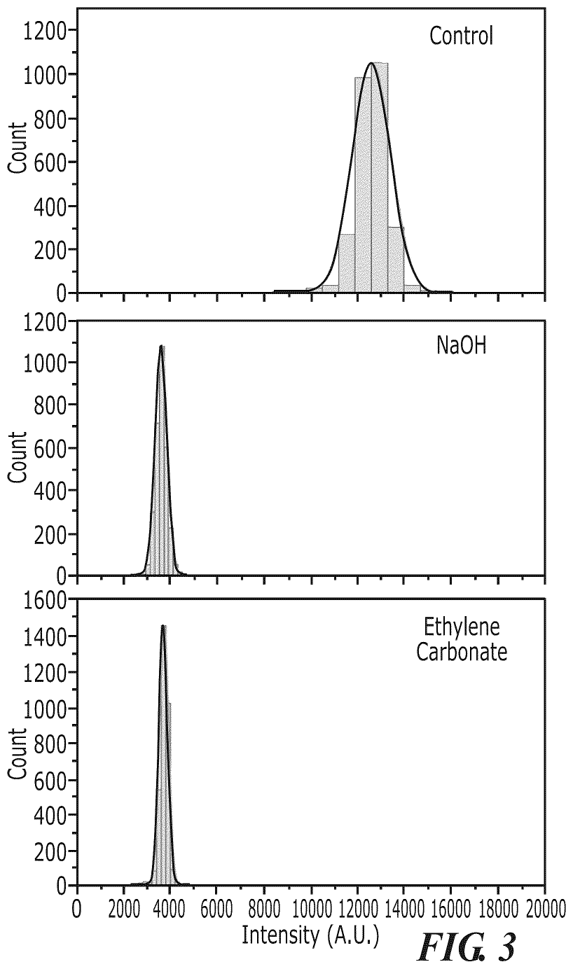

As shown in FIG. 3, these results demonstrate that 35% ethylene carbonate and 100 mM NaOH were comparably effective at denaturing the double-stranded nucleic acid molecules to generate single-stranded nucleic acid molecules.

### Example 2: Denaturing Longer Nucleic Acid Molecules in Preparation for Sequencing

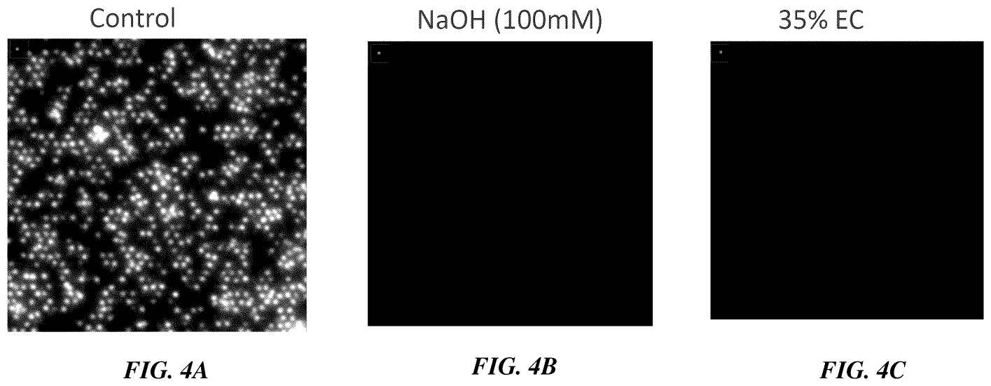

FIGS. 4A-4C shows example data on denaturation of nucleic acid molecules on a surface following treatment with ethylene carbonate (EC), NaOH (100 mM), and control, wherein nucleic acid molecules are about 200˜300 bp in length.

Sample nucleic acids were amplified using rolling circle amplification (RCA) and multiple displacement amplification (MDA) to generate DNA amplicons with lengths of approximately 200˜300 bp. The single-stranded are immobilized to a wafer. The amplicons were probed with dye-labeled primers. FIG. 4A shows an image of a coupon (a subsection) of the wafer. The amplicons on different areas (coupons) of the wafer were treated with 100 mM sodium hydroxide (NaOH) or 35% ethylene carbonate (EC), and imaged, which is shown in FIG. 4B and FIG. 4C, respectively. The coupons were imaged under identical imaging parameters, and the images are displayed in the same brightness and contrast. As seen from the lack of bright spots in the images of panels FIG. 4B and FIG. 4C, the results demonstrate that both the NaOH and EC treatments have melted off the dye-labeled primers.

Thus, this demonstrates that NaOH and EC are comparably effective at denaturing double-stranded nucleic acid molecules of relatively longer length (e.g., 200˜300 bp).

### Example 3: Hybridizing Sequencing Primers

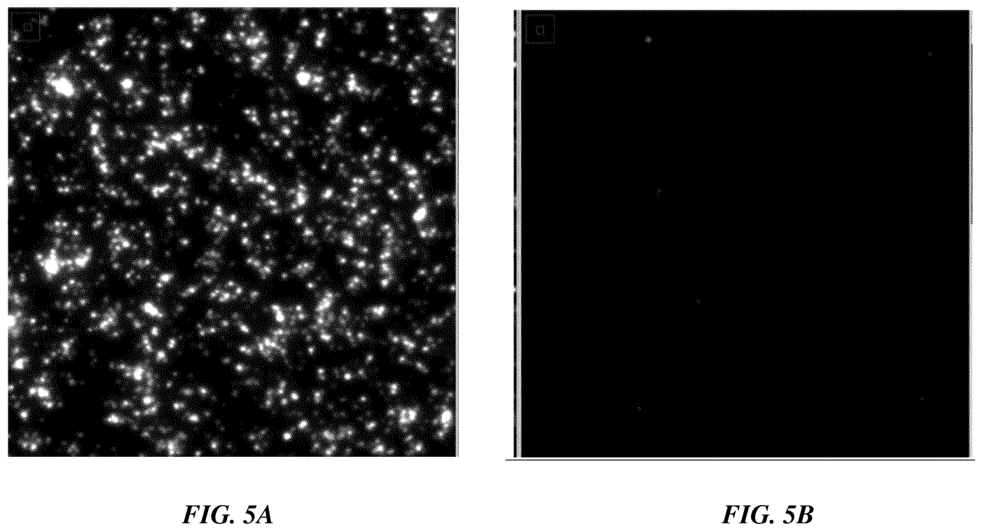

FIGS. 5A-5B show example data on hybridizing sequencing primers to amplicons in the presence of ethylene carbonate (EC) (FIG. 5A), and confirmation after NaOH treatment (FIG. 5B).

Sample nucleic acids were amplified using rolling circle amplification (RCA) and multiple displacement amplification (MDA) to generate DNA amplicons with lengths of approximately 200˜300 bp. The amplicons are immobilized to a wafer. The amplicons were treated with ethylene carbonate (EC) and without alternative duplex denaturation treatment, contacted with dye-labeled sequencing primers, and imaged. FIG. 5A shows an image of a coupon (a subsection) of the wafer after contact with the dye-labeled sequencing primers. Then, the hybridized sequencing primers were denatured via NaOH treatment, and imaged. FIG. 5B shows an image of a coupon of the wafer after NaOH treatment. It can be seen that the signals previously detected in the image of FIG. 5A are removed after NaOH treatment, which demonstrates that the sequencing primers were stripped off with NaOH treatment and thus the earlier hybridization was successful. The coupons were imaged under identical imaging parameters, and the images are displayed in the same brightness and contrast. As seen from the bright sports in the image of FIG. 5A, the results demonstrate that sequencing primers were effectively hybridized to the amplicons in the presence of EC and without an additional duplex denaturation treatment.

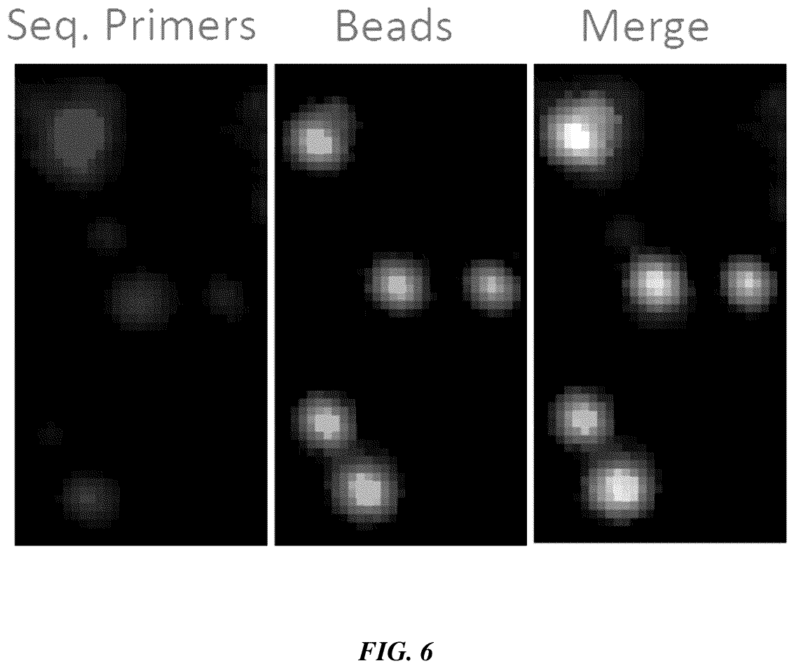

FIG. 6 shows example data on the location of sequencing primers hybridized to amplicons in the presence of ethylene carbonate (EC). The three panels in FIG. 6 display images of the same coupon. Of the three panels, the left panel displays a coupon image of signals (of pink color) from dye-labeled sequencing primers, the center panel displays a coupon image of signals (of green color) from amplicon-immobilized beads on the wafer, and the right panel displays a merged image of the two signals from the left and center panels. It will be appreciated that original color images have been gray-scaled for purposes of this patent publication, and the locations of the relatively brighter (lighter gray) signals can be distinguished from a ‘black’ background in these panel images. It is clearly seen from the merged image (right panel) that the locations of the sequencing primer and the beads overlap, which demonstrates that the hybridization of the sequencing primer is specific to the amplicons on the beads on the wafer.

### Example 4: Sequencing Nucleic Acid Molecules

This example illustrates a method for generating sequencing data associated with nucleic acid molecules (e.g., single-stranded nucleic acid molecules) that have been prepared for sequencing accordingly to the methods provided herein.

Sequencing data associated with single-stranded nucleic acid molecules is generated using a plurality of flow steps. Briefly, single-stranded nucleic acid molecules attached to a sequencing surface (e.g., a wafer) are hybridized to sequencing primers, thereby generating sequencing hybrids. A DNA polymerase is applied to the sequencing surface, and the DNA polymerase binds to the hybridized sequencing primers. A first solution containing a first plurality nucleotides (e.g., deoxy-A, deoxy-G, or deoxy-C), such as non-terminating nucleotides, and the wafer is washed to remove unincorporated nucleotides using a wash buffer. A least a portion of the nucleotides are labeled (e.g., fluorescently labeled). The presence or absence of base incorporation across the single-stranded nucleic acid molecule is detected using a fluorescence detector. This process is repeated using a second solution and a third solution, each containing a different (i.e., second and third) nucleotides to complete a flow cycle, and the flow cycles are repeated to sequence a region of the single-stranded nucleic acid molecule, or portion thereof (e.g., barcode region of the nucleic acid molecule). The solutions are separately applied to the wafer, the wafer is washed, and the presence or absence of base incorporation detected (e.g., flow signals) before applying the next solution in a cycle, for a series of cycles. The flow signals comprise a statistical parameter indicative of a likelihood for at least one base count at each flow position, wherein the base count is indicative of a number of bases of the single-stranded nucleic acid molecule sequenced at the flow position.

A method for sequencing may comprise (a) amplifying a nucleic acid molecule to generate amplicons, (b) contacting the amplicons with ethylene carbonate and a plurality of sequencing primers, to generate a plurality of single-stranded nucleic acid molecules hybridized to the plurality of sequencing primers. The method may be performed without an alternative or additional denaturation operation prior to hybridizing the sequencing primers.

What is claimed is:

1. A method of preparing nucleic acid molecules for sequencing, comprising:
contacting double-stranded nucleic acid molecules attached to a surface with ethylene carbonate to generate single-stranded nucleic acid molecules attached to the surface; and,
hybridizing sequencing primers to the single-stranded nucleic acid molecules, thereby generating sequencing hybrids.

2. The method of claim 1, wherein the ethylene carbonate has a concentration of between about 10% and about 50% volume/volume.

3. The method of claim 1 or 2, wherein the contacting is implemented at a temperature of between about 35° C. and about 50° C.

4. The method of any one of claims 1-3, wherein the double-stranded nucleic acid molecules attached to the surface are contacted with the ethylene carbonate for about 5 minutes or more.

5. The method of any one of claims 1-4, wherein the sequencing primers comprise a nucleic acid primer.

6. The method of any one of claims 1-4, wherein the sequencing primers comprise a peptide nucleic acid (PNA) primer.

7. The method of claim 6, wherein the PNA primer has increased hybridization affinity with the single-strand nucleic acid molecules compared to a nucleic acid primer.

8. The method of any one of claims 1-7, wherein a concentration of the sequencing primers is selected to favor formation of the sequencing hybrids over re-formation of the double-stranded nucleic acid molecules.

9. The method of claim 8, wherein the sequencing primers are in excess concentration compared to the single-stranded nucleic acid molecules.

10. The method of any one of claims 1-9, wherein the contacting and hybridizing occur simultaneously.

11. The method of any one of claims 1-10, wherein the double-stranded nucleic acid molecules or the single-stranded nucleic acid molecules are derived from a fluidic sample obtained from an individual.

12. The method of claim 11, wherein the fluidic sample is a blood sample, a plasma sample, a saliva sample, a urine sample, or a fecal sample.

13. The method of claim 11 or 12, wherein the fluidic sample comprises cell-free nucleic acid molecules.

14. The method of any one of claims 11-13, wherein the fluidic sample comprises DNA molecules.

15. The method of any one of claims 11-14, wherein the fluidic sample comprises cDNA molecules.

16. The method of any one of claims 1-15, wherein the double-stranded nucleic acid molecules or the single-stranded nucleic acid molecules comprise a sequencing adaptor sequence, wherein the sequencing adaptor sequence comprises a sequencing primer hybridization sequence.

17. The method of any one of claims 1-16, wherein the double-stranded nucleic acid molecules or the single-stranded nucleic acid molecules are amplification products.

18. The method of any one of claims 1-17, wherein the double-stranded nucleic acid molecules or the single-stranded nucleic acid molecules are covalently attached to the surface.

19. The method of any one of claims 1-18, wherein the double-stranded nucleic acid molecules or the single-stranded nucleic acid molecules are attached to the surface using click chemistry or amine-reactive crosslinker chemistry.

20. The method of any one of claims 1-19, wherein the surface is a bead.

21. The method of claim 20, wherein the bead is a gel bead.

22. The method of any one of claims 1-21, wherein the surface is immobilized to a wafer.

23. The method of any one of claims 1-22, further comprising attaching nucleic acid molecules in a sequencing library to the surface prior to the contacting with ethylene carbonate.

24. The method of claim 23, further comprising amplifying the nucleic acid molecules in the sequencing library attached to the surface prior to the contacting with ethylene carbonate, thereby generating sequencing colonies comprising the double-stranded nucleic acid molecules attached to the surface.

25. The method of claim 24, wherein the nucleic acid molecules are amplified isothermally.

26. The method of claim 25, wherein the amplifying occurs between about 30° C. and about 50° C.

27. The method of any one of claims 24-26, wherein the amplifying comprises one or more of rolling circle amplification (RCA), multiple displacement amplification (MDA), recombinase polymerase amplification (RPA), and molten recombinase polymerase amplification (mRPA).

28. The method of claim 27, wherein the amplifying comprises use of reagents selected from the group consisting of polymerases, recombinases, single-stranded DNA binding proteins, magnesium acetate, betaine, formamide, tetramethyl ammonium chloride, sodium dodecyl sulfate (SDS), and trimethylamine N-oxide.

29. The method of any one of claims 24-28, further comprising removing deoxyuridine primers after amplifying the nucleic acid molecules on the surface.

30. The method of any one of claims 1-29, further comprising washing the single-stranded nucleic acid molecules with a wash buffer prior to the hybridizing of sequencing primers to the single-stranded nucleic acid molecules.

31. The method of any one of claims 1-30, further comprising washing the sequencing hybrids with a wash buffer.

32. The method of claim 30 or 31, wherein the washing is repeated two or more times.

33. The method of any one of claims 30-32, wherein the wash buffer comprises tris (hydroxymethyl) aminomethane (tris), ethylenediaminetetraacetic acid (EDTA), triton, or sodium dodecyl sulfate (SDS).

34. The method of any one of claims 1-33, further comprising sequencing the single-stranded nucleic acid molecules, thereby generating sequencing data.

35. The method of claim 34, wherein the single-stranded nucleic acid molecules are sequenced using a plurality of sequencing flow steps, each sequencing flow step comprising contacting the sequencing hybrids with nucleotides, wherein at least a portion of the nucleotides are labeled, and detecting the presence or absence of an incorporated nucleotide.

36. The method of claim 35, wherein the nucleotides in each sequencing flow step comprise nucleotides of a same base type.

37. The method of claims 35 or 36, wherein the at least a portion of the nucleotides is less than all of the nucleotides in each sequencing flow step.

38. The method of any one of claims 35-37, wherein the nucleotides are non-terminating nucleotides.

39. The method of any one of claims 35-38, wherein the sequencing data comprises flow signals at the plurality of sequencing flow steps.

40. The method of claim 39, wherein the flow signals are used to determine a base count indicative of a number of bases sequenced at each flow step.

41. The method of claim 39 or 40, wherein the flow signals are used to determine a statistical parameter indicative of a likelihood for at least one base count at each flow step, wherein the base count is indicative of a number of bases of a given single-stranded nucleic acid molecule sequenced at a given flow step.

42. A method of preparing nucleic acid molecules for sequencing, comprising:
providing nucleic acid molecules attached to a surface;
amplifying the nucleic acid molecules on the surface to generate double-stranded nucleic acid molecules;
contacting the double-stranded nucleic acid molecules with ethylene carbonate to generate single-stranded nucleic acid molecules;
washing the single-stranded nucleic acid molecules with a wash buffer; and,
hybridizing sequencing primers to the single-stranded nucleic acid molecules, thereby generating sequencing hybrids.

43. The method of claim 42, wherein the amplifying is isothermal.

44. The method of claim 42 or 43, wherein the amplifying comprises one or more of rolling circle amplification (RCA), multiple displacement amplification (MDA), recombinase polymerase amplification (RPA), and molten recombinase polymerase amplification (mRPA).

45. A method of sequencing nucleic acid molecules, comprising:
providing nucleic acid molecules attached to a surface;
amplifying the nucleic acid molecules on the surface to generate double-stranded nucleic acid molecules;
contacting the double-stranded nucleic acid molecules with ethylene carbonate to generate single-stranded nucleic acid molecules;
washing the single-stranded nucleic acid molecules with a wash buffer;
hybridizing sequencing primers to the single-stranded nucleic acid molecules, thereby generating sequencing hybrids; and,
sequencing the single-stranded nucleic acid molecules using a plurality of sequencing flow steps, each sequencing flow step comprising contacting the sequencing hybrids with non-terminating nucleotides, wherein at least a portion of the non-terminating nucleotides are labeled, and detecting the presence or absence of an incorporated non-terminating nucleotide.

46. The method of claim 45, wherein the amplifying is isothermal.

47. The method of claim 45 or 46, wherein the amplifying comprises one or more of rolling circle amplification (RCA), multiple displacement amplification (MDA), recombinase polymerase amplification (RPA), and molten recombinase polymerase amplification (mRPA).

48. The method of any one of claims 45-47, further comprising generating sequencing data, wherein the sequencing data comprises flow signals detected at the plurality of sequencing flow steps.

49. The method of any one of claims 45-48, wherein the flow signals are used to determine a base count indicative of a number of bases sequenced at each flow step.

50. The method of claim 48 or 49, wherein the flow signals are used to determine a statistical parameter indicative of a likelihood for at least one base count at each flow step, wherein the base count is indicative of a number of bases of a given single-stranded nucleic acid molecule sequenced at a given flow step.
